(19) United States
(12) Patent Application Publication
(10) Pub. No.: US 2007/0218107 A1
(43) Pub. Date: Sep. 20, 2007

(54) Method of dieting and tools for implementing same

(76) Inventors: Steven A. Schnur, Miami, FL (US); Shirley DeLeon, Sunny Isles Beach, FL (US)

Correspondence Address: KIRKPATRICK & LOCKHART PRESTON GATES ELLIS LLP, 535 SMITHFIELD STREET, PITTSBURGH, PA, 15222, US

(21) Appl. No.: 11/375,155

(22) Filed: Mar. 14, 2006

(51) Int. Cl. A61K 47/00

(52) U.S. Cl. 424/439

(57) ABSTRACT

The inventive diet is designed to provide an easy and effective meal plan to assist an individual to meet his/her dietary goals. The dieter consumes about four meals per day, wherein the total daily caloric content of the meals comprises about 45% carbohydrates selected from fiber rich foods containing at least about 2 grams of dietary fiber for each 90 calorie serving of said fiber rich food, about 25% lean protein and about 30% heart healthy fats. The recommended menus are simple, interchangeable and realistic and do not deprive the dieter of essential nutrients.

### BACKGROUND

The present invention relates to a method of dieting and weight management that relies on the implementation of simple rules for the dieter but preserves sound nutritional principles.

Notwithstanding the recognition of the problems it creates, obesity remains one of the most serious threats to health in the United States and other developed nations. Excessive weight has been implicated in coronary disease, the growing incidence of Type II diabetes and a wide variety of stress induced injuries to the body, such as injuries to the knees, hips and the lower back. In addition, excessive weight discourages physical activity. Reduced levels of physical activity further exacerbate the deleterious health effects of obesity.

Not surprisingly, a great deal of effort has gone into addressing the problems created by excessive weight. Fad diets, diet pills, diet foods, liposuction, intestinal bypass surgery, and lifestyle changes have all been advanced as solutions to the problem of overweight, some with greater success than others.

By far, the most popular approach to addressing overweight is dieting. The classical approach of simply reducing caloric intake can be effective in promoting weight loss. However, natural metabolic effects cause hunger pains, cravings and the like which have a negative impact on successful weight reduction. In addition, habitual eating, for example, out of boredom, for pleasure, or to ease tension or depression tend to sabotage successful dieting. If reducing caloric intake can be implemented and rigorously maintained for an extended period of time, for example on the order of one to two years, changes in metabolism, habits and associated characteristics can make the weight loss effective in the long-term.

Given the level of public concern about overweight and the preference for treating the problems of overweight with diet, numerous dietary options have evolved. Many people still adhere to diets involving control of caloric intake. Others follow the options provided by various low-carbohydrate diets, all protein diets, vegetarian diets, low fat diets, and other approaches that restrict or even eliminate one or more food groups.

Notwithstanding the numerous incentives for weight loss and the numerous approaches to resolving this problem, statistics reveal that obesity is on the rise and the health problems associated with obesity, together with the high costs of care to treat those problems, is an increasingly serious concern for everyone.

Low fat diets substantially exclude cheeses, whole milk, nuts, sour cream, salad dressings, sauces, butter and desserts. Not surprisingly, such diets are not very appealing and have not met with widespread, and more to the point, long term, acceptance by dieters. In addition, important nutritional components found in many fats, such as the fats in nuts and olive oil, are excluded.

Low-calorie diets, which tend to emphasize vegetables, salads, fat-free milk, cereals and similar foods, often fail to satiate a dieter's sensation of hunger. Nevertheless, such diets do have much to recommend themselves in their substantial nutrient value and vitamin content, but pose serious nutritional issues due to the limited amount of vitamins consumed that are found in fruit and animal sources of protein, such as meat and fish.

More recently, substantial success has been achieved with low carbohydrate diets. Low carbohydrate diets seem to work, at least at the start from the standpoint of losing weight, but the nature of foods consumed may be problematic and many dieters find it impossible to maintain the low carbohydrate lifestyle. In one such diet, people are encouraged to consume large quantities of low carbohydrate foods, but are also counseled that they can eat unrestricted amounts of fats. Foods such as butter, oil, beef, chicken, eggs and fish are eaten with little or no limits. Potatoes, breads, fruit, pasta and the like must be avoided in order to make this diet effective. While this diet incorporates of green vegetables, it excludes vegetables with any sugar content, such as carrots, corn, beets, and peas, as well as grains and fruits, all good sources of nutrients.

Perhaps more seriously, many attractive low carbohydrate foods, such as beef, shrimp, butter and like have high levels of cholesterol, and therefore, present a substantial risk of coronary artery disease to significant segments of the population.

Generally, diets that focus on one or two food groups to the exclusion of others gain popularity for a time and may even produce good results for the short term, but suffer from lack of variety of food types, and can pose health issues to some people over the long term. They are all too often abandoned in frustration because people can not refrain from eating an entire food group for long periods. Nevertheless, experience indicates that many people with relatively diverse and healthful diets maintain reasonable weights and health. While, to some extent, their good health may be a function of genetic characteristics, genetic factors are not believed to play a significant role in most members of the general population.

There is a need for a diet regimen that will provide a nutritionally balanced, healthful and psychologically satisfying way to lose weight and thereafter, maintain weight at a healthy level.

### SUMMARY

The present invention focuses on maintaining variety in the types of foods consumed and offers sufficient quantities of food to be physically and psychologically satisfying and filling. At the same time, the diet components are selected for their health-supporting characteristics. Finally, the inventive method provides a simple formula for measuring meals which is convenient to use, while at the same time provides a great deal of flexibility in the selection of foods. Moreover, the same is achieved without the exclusion or severe minimization of any food group, thus accommodating a wide range of tastes.

The inventive diet offers a method of weight management comprising one or both of weight loss and target weight maintenance. The method comprises consuming a plurality of meals per day, wherein the total daily caloric content of the meals comprises about 41 to 49% carbohydrates selected from fiber rich foods containing at least about 2 grams of dietary fiber for each 90 calorie serving of said fiber rich food. The total daily caloric content of the meals may also comprise about 22 to 28% protein selected from lean protein sources having less than about 5 grams of fat per ounce of the lean protein source. The total daily caloric content of the meals may also comprise about 26 to 34% fats selected from monounsaturated and polyunsaturated fats.

More particularly, in accordance with the invention, 45 percent of the dieter's total daily calories come from low-fat, fiber-rich carbohydrates, 25 percent of the total daily calories come from lean protein, and 30 percent of the total daily calories come from foods containing heart healthy fats.

In accordance with the present invention, caloric intake is initially set at about 1400 calories per day for women and 1800 calories per day for men. Adjustments in caloric intake may be made if the dieter is losing more or less than the recommended amount of weight on a weekly basis. In accordance with the invention, fiber intake is preferably about 25 grams per day for an average woman and about 35 grams per day for an average man. That amount of fiber is twice that of the four major diets currently in vogue, namely the Atkins™ diet, the South Beach® diet, the Weight Watchers® diet and the Zone™ diet.

The inventive method of dieting uses one menu format for men and another for women due to the different caloric maximums each group can consume.

The total daily caloric intake for men may initially include five servings of starch, two servings of fruit, two servings of milk or yogurt products, four servings of fats, two servings of primary proteins, two servings of secondary proteins and two servings of vegetables. The total daily caloric intake for women may initially include four servings of starch, two servings of fruit, two servings of milk or yogurt products, three servings of fats, two servings of primary proteins, one serving of secondary proteins and one serving of vegetables. The inventive diet encourages men and women to additionally select up to four dividend foods selected from foods and beverages substantially devoid of caloric value, foods and beverages in quantities sufficiently small that such foods and beverages are substantially devoid of caloric value, fat free condiments, water and combinations thereof, as described more fully below. Adjustments to the number of servings may be made if the individual is losing more or less than the recommended two pounds per week.

The inventive diet includes starches (bread, potatoes, rice, etc.), meats, fish, green vegetables, oils, poultry and other foods. Classic desserts, such as chocolate cake, ice cream, and the like can be eaten in limited quantities. More specifically, dieters eat foods from seven distinct food groups: primary proteins, secondary proteins, milk or yogurt products, starches, fruits, vegetables and fats. Each serving of food is in about 90 calorie portions. Four meals should be consumed each day. The result is a diet which has great diversity and appeal.

Thus, in accordance with the present invention, dieters eat a variety of fruits, vegetables, proteins and starches every day in a diet that is not low carbohydrate, low-fat, or high-protein. Rather, the diet is balanced and based on sound nutritional principles, making it more likely to be adhered to by dieters over an extended period of time.

In accordance with the invention, dieters are given a simple rule to assess carbohydrates. There must be at least about 2 grams of dietary fiber in each 90 calorie serving of starchy foods, referred to herein as the 2:90 Rule. The 2:90 Rule applies to starches, vegetables and fruit. Milk and yogurt products would generally not be included unless combined with added fiber, as recently developed by food manufacturers. Whole-grain foods, which have substantial amounts of vitamins, minerals and other nutrients, are encouraged. By adherence to this Rule, a wide range of starchy foods may be integrated into a diet consisting additionally of meats, seafood, vegetables, fruit, milk or yogurt products and oils or other fats. Dieters are instructed by their nutritionists, health care providers or counselors, to use about three and four servings, respectively for women and men, of heart healthy fat, and to consume the balance of their caloric intake of the day in lean, protein rich foods.

For best results, compliance with the dietary regimen includes some amount of physical activity. A recommended level of activity would be enough to bum at least 1500, and preferably 2000 calories per week.

The inventive method provides a method of counseling an overweight individual regarding weight loss and weight management, including teaching the individual to consume at least four meals each day of the component foods in a recommended ratio of carbohydrates to proteins to fats, teaches which proteins are acceptable proteins to achieve a desired weight objective, teaches which fats are acceptable fats to achieve a desired weight objective, and teaches which carbohydrates are acceptable to achieve a desired weight objective.

The teaching regarding acceptable proteins may comprise teaching the use of lean proteins. The teaching regarding acceptable fats may comprise teaching the use of heart healthy fats. The teaching regarding acceptable carbohydrates may comprise teaching the consumption of carbohydrates which, for every 90 calorie portion, contain at least about 2 grams of fiber.

The inventive method may be implemented by means of printed publications, informative and preferably interactive websites and combinations thereof and by direct personal or group counseling by healthcare providers, nutritionists, therapists, teachers and counselors.

### DESCRIPTION OF THE DRAWINGS

The inventive diet may be understood by reference to the exemplary meal plans shown in the accompanying figures. While not mandatory, the meal plans shown in the following figures may be used as shown, or as merely a guide to daily distribution of food servings. Individuals may, on their own or with the assistance of a counselor, health care provider, nutritionist or others, design meal plans falling within the scope of the inventive diet using the equivalents provided herein.

FIG. 1 is a form which may be used by a female dieter during the first two weeks of the inventive diet, and which lists the portions of foods from various categories which are consumed in accordance with the inventive diet.

FIG. 2 is a form which may be used by a female dieter during weeks three and four of the inventive diet, and which lists the portions of foods from various categories which are consumed in accordance with the inventive diet.

FIG. 3 is a form which may be used by a female dieter during weeks five and six of the inventive diet, which lists the portions of foods from various categories which are consumed in accordance with the inventive diet.

FIG. 4 is a form which may be used by a female dieter during weeks seven and eight of the inventive diet, which lists the portions of foods from various categories which are consumed in accordance with the inventive diet.

FIG. 5 is a form which may be used by a male dieter during the first two weeks of the inventive diet, which lists the portions of foods from various categories which are consumed in accordance with the inventive diet.

FIG. 6 is a form which may be used by a male dieter during weeks three and four of the inventive diet, which lists the portions of foods from various categories which are consumed in accordance with the inventive diet.

FIG. 7 is a form which may be used by a male dieter during weeks five and six of the inventive diet, which lists the portions of foods from various categories which are consumed in accordance with the inventive diet.

FIG. 8 is a form which may be used by a male dieter during weeks seven and eight of the inventive diet, which lists the portions of foods from various categories which are consumed in accordance with the inventive diet.

### DETAILED DESCRIPTION

Heart healthy fats, as used herein, means monounsaturated fats and polyunsaturated fats.

Low fat, as used herein, means 5 grams of fat or less per one ounce serving of protein or milk or yogurt products, excluding fats themselves.

Lean protein, as used herein, means protein having about 5 grams or less of fat per each one ounce serving. Lean proteins having from about 3 to about 5 grams of fat per ounce are referred to herein as secondary proteins.

Very lean protein, as used herein, means protein having about 3 grams of fat or less per each one ounce serving. Very lean proteins are referred to herein as primary proteins.

Fiber rich, as used herein, means about two grams of dietary fiber for approximately every 90 calorie serving of starches.

Dietary fiber, as used herein, means plant materials which are not digested in the human stomach and small intestine. Dietary fiber comprises structural and storage polysaccharide's of plant cells as well as lignin.

Starches, as used herein, means grains and starchy vegetables.

Caloric content or caloric value, as used herein, means that the food provides energy when metabolized conforming to the recognized understanding of the term, “Calorie” or “Calories”; that is the quantity of heat needed to raise by one degree Celsius the temperature of a kilogram of water. For example, carbohydrates have four Calories per gram, fats have nine Calories per gram and proteins have four Calories per gram. The term calorie or Calorie, as used herein, refers to the recognized understanding of the term “Calories” for food, as stated above, regardless of whether or not the word is capitalized.

Substantially devoid of caloric value, as used herein, means that the total daily caloric value of the dividend foods or beverages is 80 calories or less. For example, each portion of a dividend food may have 15-20 calories or less such that consuming the three to four dividend foods recommended per day results in consumption of 80calories or less in dividend foods for that day. Alternatively, any combination of three or four of such dividend foods can have up to about 80 calories. Alternatively, the three or four dividend foods of 80 calories or less can be consumed in one meal in a given day.

The inventive diet may be taught to individuals in need of counseling in weight management, including weight loss to achieve a target weight objective and maintenance of a desired weight range. Counselors may be health care providers, such as physicians or nurses, nutritionists, therapists, life coaches, physical trainers, exercise physiologists, teachers and others. The inventive diet recommends an ideal daily caloric ratio of carbohydrates to proteins to fats at 45:25:30. In other words, 45 percent of the calories consumed daily should come from fiber rich carbohydrates, 25 percent of the calories consumed daily should come from lean protein, and 30 percent of the calories consumed daily should come from heart-healthy fats. Of course, some variation from the recommended ratio is likely, if not inevitably, to be encountered for numerous reasons, including an individual's metabolic characteristics, differences in actual caloric value of foods compared to label-listed values or particular food choices or portion measurements. However, adherence to a daily caloric ratio of carbohydrates, proteins and heart healthy fats approaching the recommended 45:25:30 ratio, together with adherence to other dietary rules and recommendations will still lead to weight loss and improved weight management while satisfying nutritional needs.

The inventive method of weight management comprises consuming foods having a limited caloric content. An individual following the inventive method eats, or a weight management counselor instructs the individual to eat, a first group of foods comprising low fat and fiber rich carbohydrates in an amount comprising from about 41 to 49% of the total calories consumed per day, but excluding in the first group of food calories from fat. The individual consumes, or is instructed by the counselor to consume, a second group of foods comprising lean protein in an amount comprising about 22 to 28% of total calories per day consumed, but excluding in the second group of foods calories from fat. The individual also consumes, or is instructed by the counselor to consume, heart-healthy fats in an amount comprising about 26 to 34% of total calories consumed per day.

A significant part of the inventive method includes teaching the individual to follow a rule governing selection of carbohydrates. The carbohydrate selection rule provides that for every 90 calorie portion, a carbohydrate should contain at least about 2 grams of fiber.

Although qualifying carbohydrates must pass the 2:90 Rule, foods with higher fiber content per 90 calorie serving may be consumed. On average, higher fiber content in the overall carbohydrate portion of the diet is likely on account of natural tendencies toward variety. For example, qualifying carbohydrates would include such diverse products as cauliflower (9 grams of fiber per 90 calories serving), potatoes, oranges (four grams of fiber per 90 calorie serving), spinach (about 8.5 grams of fiber per 90 calorie serving), raw apricots (about 3.5 grams per 100 calorie), asparagus (about 9 grams), mangoes (about 2.5 grams), fresh sweet peppers, beans, rye crackers (about 4 grams), and many that are not typically thought of as high fiber, meet the 2:90 Rule. An example is Breyers® brand, 98 percent fat-free ice cream, which qualifies as an acceptable carbohydrate under the 2:90 Rule because it has about 3.5 grams of fiber per 100 calorie serving.

In accordance with the inventive approach where many foods may have relatively high fiber content, it is expected that dieters will easily be able to achieve the recommended 20-35 grams of fiber per day.

Other foods, such as pizza, and apple strudel do not qualify. Also, somewhat surprisingly, a typical 270 calorie oat bran muffin has only 4.6 grams of fiber, so therefore, does not qualify for inclusion in the inventive diet.

Thus, unlike conventional dieting approaches which remove entire categories of foods and the nutrients associated with them, the present method of dieting preserves variety, vitamin and nutrient diversity, and at the same time increases fiber content.

The importance of fiber combined with a diversified food selection for psychological satisfaction and good nutrition should not be overlooked. Besides being filling, fiber has been demonstrated to be important for a variety of reasons. Inclusion of viscous dietary fibers in the diet appears to lower blood cholesterol levels and normalize blood glucose and insulin levels. Thus, viscous fibers will have the effect of treating cardiovascular disease and type II diabetes. In addition, while a recent study appeared to indicate that a fiber rich diet did not slow the formation of precancerous polyps in the colon; numerous studies have shown that a fiber rich diet is associated with a lower risk of colon cancer. Some fiber rich products, such as oat bran and psyllium husk, have been shown to be effective in the reduction of cholesterol when included within a diet. It would appear that such cholesterol-lowering fibers work by interfering with bile acid absorption in the ileum, perhaps, based on changes in the composition of the bile acid flow after consumption of viscous-fiber containing foods, thus dampening cholesterol synthesis. There are other advantages to fibrous foods which typically include nonnutritive components such as phytoestrogens, and antioxidants.

All fibers are not the same. Presently, there is much use of the terms soluble and insoluble fiber. However, such descriptions do not appear to be as informative as other terms and, accordingly, these terms are being abandoned in the literature. Generally, dietary fiber comprises structural and storage polysaccharide's of plant cells as well as lignin. Lignin is a polyphenylpropane construct and is a very small part of the human diet. Fibers are plant materials which are not digested in the human stomach and small intestine. Rather, microflora in the large intestine work to a least partially ferment the fibers, thus promoting normal Taxation and are thus useful in the treatment of diverticulosis and diverticulitis. In addition, fiber may prevent duodenal ulcers and cancer in the gastric cardiac region. The high fiber content of the present invention thus has a host of advantages. For purposes of the invention commonly listed fiber amounts on foods may be employed.

The above ratio gives the dieter enough carbohydrates to prevent ketosis and spare protein breakdown. Following the inventive diet can be expected to result in a healthy weight loss of approximately two pounds per week in an overweight individual. The relative distribution of food types and the quantity of food recommended has been found to be psychologically and physically satisfying. Thus, the inventive diet is easy to follow, because it allows the dieter to eat a satisfying amount of his/her favorite starches like pasta, rice, cereal, bread, and potatoes, all of which are heavily restricted in conventional low-carbohydrate diets.

While 45% of the total calories consumed per day of carbohydrates are the target amount, some variation is permitted. For example, at least 41% -49%, and preferably 42% -48%, and more preferably 43-47%, and most preferably 44% -46% of the total daily caloric consumption of carbohydrates in the inventive diet come from starches, such as pasta, rice, breads and cereals. Whole-grain products are preferred.

As noted above, about 25% of the inventive diet's total daily calories come from lean protein sources, which allow the dieter to feel full and satisfied. Again, while 25% of the daily caloric total should include lean proteins, some variation may be tolerated. For example, 22% to 28%, and preferably 23% to 27% and more preferably 24% to 26% of the inventive diet's total daily calories come from lean protein sources, such as skinless chicken or turkey breast, goat meat, haddock, fat free cheese and cooked beans. Other lean proteins are shown in Table 12.

Finally, about 30% of the inventive diet's total daily calories come from “heart healthy” monounsaturated and polyunsaturated fats. While 30% of the daily caloric total should include heart-healthy fats, some variation may be tolerated. For example, 26% to 34%, and preferably 27% to 33% and more preferably 28% to 32% of the inventive diet's total daily calories come from heart-healthy fats, such as olive oil or canola oil and other fats shown in Table 7.

The person dieting may include some dividend foods in the daily meal plan. Dividend foods are shown in Table 8. The inventive method encourages men and women to additionally select up to four dividend foods, and preferably from three to four dividend foods, per day selected from foods and beverages substantially devoid of caloric value, foods and beverages in quantities sufficiently small that such foods and beverages are substantially devoid of caloric value, fat free condiments and combinations thereof, as described more fully below.

The person dieting may liberally partake in a group of foods that consist of foods having no or minimal nutritive or caloric value, referred to herein as Free Foods. Many of these foods have relatively high flavor content and/or are customarily eaten in small quantities and thus, may help to address psychological cravings. Exemplary free foods are shown in Table 13.

The inventive diet is portion based. Portion control is an important factor in successful weight loss. In accordance with the inventive diet, the dieter may consult the exemplary equivalents shown in Tables 1-12 to determine the appropriate portion size to achieve about 90 calories per serving of any of the seven food groups of the diet. The dieter therefore does not have to count calories at each meal, but only has to follow the portions that are equivalent to the 90 calorie servings. For foods that are not set forth on the list, the calorie content can be determined from the food label or from available publications of the caloric content of a variety of different foods.

The inventive diet recommends that the dieter stays active. An individual is counseled to engage in activity sufficient to bum at least about 1500 calories, and preferably about 2,000 calories, on average per week through exercise. Exemplary activities exercises are shown in Table 14. A weekly activity planner listing physical activities may be given to the dieter. Such activity planner can list various activities that will bum the recommended 1500 to 2000 calories per day.

Since men and women have very different nutritional needs from the standpoint of the quantity of food, the inventive diet provides additional portions for men.

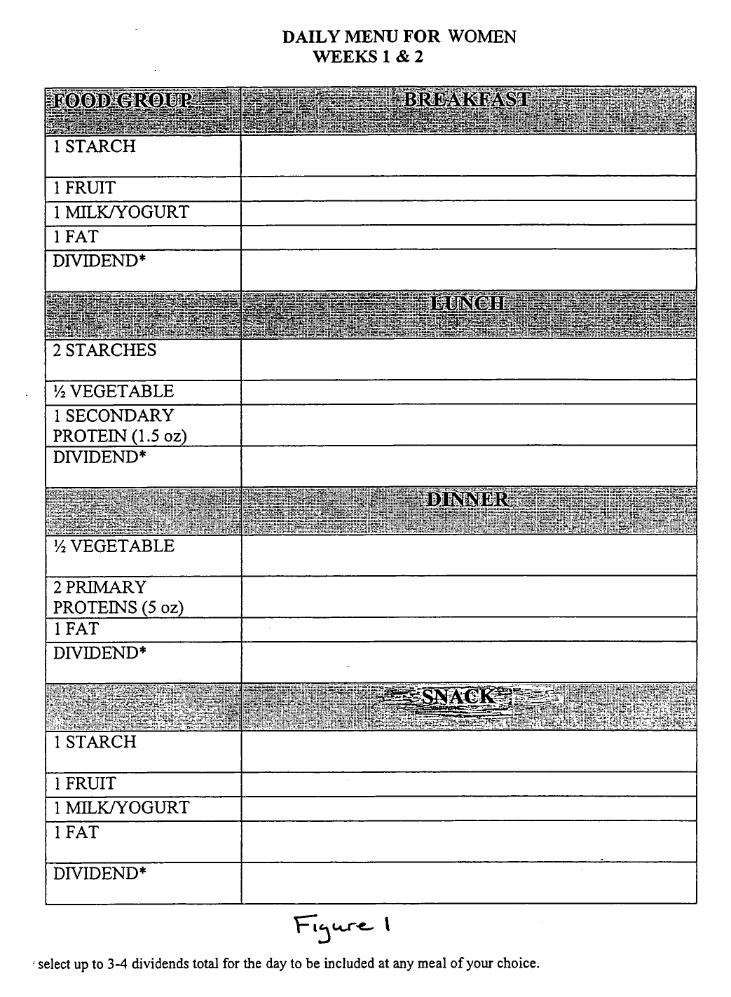

Generally, in order to implement these different diets for men and women, the dieter is provided with a set of forms (for example, comprising paper or computer forms) to be is to generate daily meal plan. One of such forms is illustrated in FIG. 1. Separate sets of forms are provided for both men and women.

Generally, each day the plan includes four meals: breakfast, lunch, dinner, and dessert or a healthy snack. The dieter can choose from among the following seven food groups: starch, fruit, milk or yogurt, fat, vegetables, primary proteins, secondary protein. In addition, a dividend, or treat, can be accommodated within the structure of each meal of the inventive diet.

The inventive diet also recommends several meals each day. In one embodiment, up to four meals each day are encouraged to accommodate normal digestive cycles. In a preferred embodiment of the method of weight management, the individual dieter is instructed to eat a variety of healthy foods adding up to four meals each day: breakfast, lunch, dinner and dessert or a snack. For dessert, the dieter can use either the recipes provided herein or use appropriate portions of foods from four food groups as a snack. It is also important that the dieter drink adequate amounts of water throughout the day, approximately six to eight 8-ounce glasses or more. For every 25 pounds over his or her recommended weight, the dieter should drink an additional 8 ounces of water over the recommended eight 8-ounce portions of water.

For starch selection, the dieters select from what may be characterized as “safe” starches. The inventive diet allows the dieter to select starches that provide at least 2 grams of fiber for every 90 calorie serving. This is a simple way for dieters to select starches which are effective and healthy in the context of the inventive diet, by providing adequate amounts of dietary fiber. Dieters are directed, in accordance with the inventive diet, to apply this rule to a variety of products. The 2:90 Rule is not intended to be applied to commercially available low carbohydrate products, which should be generally avoided because many have added amounts of protein and fat. Use of those products would result in the consumption of too much protein and fat, beyond the recommended daily percentages of proteins and fats.

More particularly, a woman beginning the inventive diet would begin weeks one and two of the diet by following the diet outlined in blank form in FIG. 1. For breakfast, she would have a portion of a starch, a fruit, milk or yogurt, a fat, and a dividend or treat. For lunch, she may have two servings of a starch, one half serving of vegetables, one secondary protein and one dividend food. For dinner during the first two weeks of the diet, the female dieter may have another half serving of a vegetable, two servings of a primary protein, one serving of a fat and one dividend food. As a treat or fourth meal, the female dieter may have one serving of a starch, one fruit serving, one serving of milk or yogurt, one fat serving and one dividend food.

The starch may be selected from a wide range of options selected from one of the grain portions of Table 1, one of the starchy vegetable portions of Table 2, one of the other starch portions listed in Table 3, or one of the flexibility starch portions listed in Table 4. The product specific shopping list in Table 4 is provided to identify some exemplary starches that meet the 2:90 Rule. The list is representative and may be used as a guide. It is not an exhaustive list. The nutritive content of commercially available foods may vary over time, so the dieter is cautioned to read labels. It is noted that in accordance with the invention, generally, fractional parts of different portions may be combined to make a portion.

Table 1 is a list of grains the dieter can select from for the daily menu. All starches must meet the 2:90 Rule.

TABLE 1
2:90 SAFE ZONE STARCHES
One starch can be substituted for one-ninety calorie
serving of any of the following:

Light bread
Regular bread
Pita
Bagels
Dry Cereals (calcium fortified)
Cereal bars
Hot Cereals
Toasted wheat bran
Wheat germ
Oat bran
Flatbread

Table 2 is a list of starchy vegetables the dieter can select in accordance with the forms of the figures for the daily menu.

TABLE 2
Starchy Vegetables
The following amounts are equivalent to approximately 90-calories.

½ cup corn
1 medium ear of corn (8″ long)
½ cup green peas
1 small sweet potato, plain with skin (5″ long)
1 small potato, plain, baked with skin (5″ long)
1 cup baked winter squash (acorn, butternut, pumpkin, turban, spaghetti,
golden, nugget, Red Kuri, Kabocha, heart of gold, sweet dumpling,
or Delicata)
⅔ cup mixed vegetables, with corn & peas
½ cup plantain

Table 3 is a list of all other “safe” starches the dieter can select for the daily menu. “Safe” starches as used herein means starches which have at least 2 grams of fiber for a 90 calorie serving.

TABLE 3
Other Starches
The following amounts are equivalent to approximately 90-calories.

¼ cup baked beans, vegetarian style
⅓ cup Pasta/Noodles: must be cooked al dente and not overcooked.
One ounce of dry pasta is ⅓ cup cooked.
½ cup Pasta Sauce*
¾ cup Soup (bean, mixed vegetables, or homemade soup containing
pasta (that meets 2:90 rule) or rice (that meets 2:90 rule).
*The best sauce in a jar will provide about 3 grams of fiber and no saturated fat for every ½ cup serving. The dieter may be counseled to try to select sauce that contains fewer than 400 mg of sodium per serving if possible. If high blood pressure is a problem, the dieter may be counseled to make his or her own sauce where the ingredients can be better controlled.

To aid the dieter in selecting appropriate foods when shopping, an exemplary list showing the fiber content of various commercial products are shown in the table below. These are safe starchy foods having at least 2 grams of fiber per approximate 90 calorie servings, and may be used in accordance with the inventive diet.

TABLE 4
Shopping List
I. Any of the following starches meet the 2:90 Rule and count as one serving.

BREADS
1 slice Arnold Hearty Classics ® 100% whole wheat (3 g fiber)
2 slices of Holsum ® light white (5 g fiber)
2 slices of Holsum ® light Italian (5 g fiber)
2 slices of Holsum ® light wheat (4 g fiber)
2 slices Nature's Own ® Healthline Double Fiber Wheat (10 g fiber)
2 slices of Nature's Own ® Healthline light honey wheat (5 g fiber)
1 Pepperidge Farm ® whole wheat mini bagel (3 g fiber)
1 slice Pepperidge Farm ® Pumpernickel (80 cal, 2 g fiber)
1 slice of Nature's Own ® 100% whole wheat (3 g fiber)
1 slice Pepperidge Farm ® Natural Whole Grain Multi-grain bread (3 grams fiber)
2 pieces of All Natural New York flatbreads ® (2 g fiber)
½ cup Ian's ® Panko breadcrumbs, no artificial colors, flavors or preservatives
1 muffin Thomas ® Light Multi-Grain English Muffin (100 cal, 8 gm fiber)
1¼ loaves Toufayan Bakeries ® Pitettes Whole Wheat Pita Bread (2 g fiber)
2 slices Wonder ® Light Wheat (40 cal each, 5 g fiber)
2 slices Wonder ® Light Italian (40 cal each, 5 g fiber)
1 slice Wonder ® Stone Ground Whole Wheat (80 cal, 2 g fiber)
2 slices Weight Watchers ® Whole Wheat (45 cal each, 4 g fiber)
1 Weight Watchers ® bagel (150 cal for whole bagel, 9 g fiber) - Use ½ (75 cal, 4.5 g fiber)
DRY CEREALS
½ c Cheerios ® (90 cal, 2.5 g fiber)
½ c Chex ® Multi Bran Whole Grain (95 cal, 3.5 g fiber)
⅔ c General Mills ® Fiber One Honey Clusters (7 g fiber)
⅔ c General Mills ® Fiber One (18.7 g fiber)
½ c Grape Nuts ® Flakes (90 cal, 2 g fiber)
½ c Heart to Heart Kashi ® (90 cal, 3.5 g fiber)
½ c Kashi ® Go Lean (6.6 g fiber)
½ c Kashi ® Good Friends (85 cal, 6 g fiber)
⅓ c Kelloggs’ ® All Bran bran buds (13 g fiber)
¾ c Kelloggs’ ® All Bran extra fiber (21 g fiber)
½ c Kelloggs’ ® All Bran original (10 g fiber)
¾ c Kelloggs’ ® All Bran Apricot Bites (6 g fiber)
¾ c Kelloggs’ ® Complete wheat bran flakes (5 g fiber)
½ c Raisin Bran ® (95 cal, 4 g fiber)
½ c Total ® Flakes Whole Grain (90 cal, 2 g fiber)
¾ c Quaker ® Crunchy corn bran (5 g fiber)
½ c Uncle Sam's ® (95 cal, 5 g fiber)
⅓ cup fruit muesli
HOT CEREALS
⅓ c Quaker ® oat bran hot cereal (3.6 g fiber)
2.5 Tbsp Wheatena ® toasted wheat cereal (2.5 g fiber)
⅔ c Kretschmer ® toasted wheat bran (18.5 g fiber)
3 Tbsp Kretschmer ® wheat germ (3.2 g fiber)
¾ packet Quaker ® instant oatmeal lower sugar apples & cinnamon (2.3 g fiber)
¾ packet Quaker ® instant oatmeal regular flavor (2.3 g fiber)
¼ c dry of Old Fashioned Quaker ® Oats (2.1 g fiber)
CEREAL BARS
1¼ bars Health Valley ® Date Bakes Fat Free (3.9 g fiber)
WAFFLES
1 Kashi Go Lean ® original waffles or blueberry (3 g fiber)
TORTILLAS
1 La Real whole wheat tortillas (2 g fiber)
1 La Banderita Soft Taco Low Carb (95 cal, 5 g fiber)
1 La Banderita Soft Taco Fat Free Flour Tortilla (85 cal, 4 g fiber)
CRACKERS/TOAST/CRISPBREAD
2.5 crackers Natural Ry Krisp ® fat free (5 g fiber)
2.5 crackers Sesame Ry Krisp ® (3.75 g fiber)
5 pieces Old London ® Melba toast whole grain or sesame (3.3 g fiber)
2.5 slices Wasa ® Fiber Rye crispbread (5 g fiber)
2 slices RyVita ® dark or light rye whole grain crispbread (3 g fiber)
2 pieces of Kavli ® 5 grain crispbread (4 g fiber)
1 matzo Manischewitz ® whole wheat (3 g fiber)
¾ matzo Streit's ® Matzos 100% whole wheat (2.9 g fiber)
GRANOLA
¼ cup granola, the original (2.3 g fiber)
FLOUR
⅕ cup whole wheat flour (3.3 g fiber)
POPCORN/RICE CAKES
4 cups popped of Jolly-Time ® Healthy Pop butter flavor 94% fat free (8 g fiber)
4 cups popped of Jolly-Time ® Healthy Pop Popcorn, caramel apple 94% fat free (4 grams
of fiber)
4 cups Organic no oil added microwave popcorn
4 cups Organic no salt no oil microwave popcorn
4 cups popped Smart Balance Popcorn (4 g fiber)
4 slices of Real Foods ® Corn Thins Multigrain (2 g fiber)
3 Orville Redenbacker's ® rice cakes white cheddar cheese (3 g fiber)
8 Orville Redenbacker's ® minicakes sour cream & onion (2 g fiber)
RICE/OTHER GRAINS
1-cup dry rice makes 3 cups cooked.
⅓-cup cooked Fantastic ® Organic Whole-wheat couscous (3 g fiber)
⅓ cup cooked Ancient Harvest ® Organic Quinoa Inca Red (2 g fiber)
⅓ cup cooked Heartland ® America's Original Bulgur wheat (3.5 g fiber)
¼ cup cooked Kashi ® Breakfast Pilaf (3 g fiber)
¼ c cooked Kashi 7 whole grain pilaf (3 g fiber)
⅓ cup cooked Lundberg ® Wild Blend gourmet blend of wild & premium brown rice
⅓-cup cooked whole oat groats
⅓ cup cooked roasted buckwheat (Kasha)
⅓ cup cooked Barley, pearled
⅓ cup cooked Barley, hulled
⅓ cup cooked Hard red winter wheat
⅓ cup cooked Soft wheat berries
2 Tbsp dry mix Natural ® Tabouli wheat salad mix
PASTA
1 oz of dry pasta is approximately ⅓ cup cooked.
⅓ c cooked Organic pasta bionaturae ® 100% stone ground whole durum wheat chiocciole
(2.5 g fiber)
⅓ c cooked De Boles ® organic whole wheat angel hair or spaghetti (2 g fiber)
⅓ c cooked De Boles ® wheat free corn spaghetti style (2 g fiber)
⅓ c cooked De Boles ® organic whole-wheat spaghetti style pasta (2 grams fiber)
⅓ c cooked Hodgson Mill ® certified organic spaghetti whole wheat gourmet pasta with
milled flax seed (2.3 g fiber)
⅓ cup cooked Hodgson Mill ® certified organic fettuccine (whole wheat gourmet pasta
with milled flax seed) (3 grams fiber)
⅓ c cooked Hodgson Mill ® whole wheat bow tie or whole wheat spinach spaghetti (2.3 g
fiber)
⅓ c cooked Vita Spelt ® whole grain organic spaghetti (2.2 grams fiber)
½ c pasta sauce home-made (with fresh tomatoes) no salt or in jar that meets rule*.
CHIPS/OTHER
8 chips Garden of Eatin’ ® Black Bean all natural tortilla chips (2.6 g fiber)
10 chips Low fat gourmet baked potato chips Kettle Krisps ®
4 Tbsp hummus (2 g fiber)
Other Dessert Starches
⅓ cup Fruit Muesli
¼ cup granola, the original, with no added fruit or sugar (2.3 g fiber)
1 oz (approx. 4 crackers) Rosario's All Natural Fat free cinnamon flatbread crackers
¾ bar Fig Newtons ® 100% whole grain bars by Nabisco
2 Fig Newtons ® cookies 100% whole grain
3 cookies Health Valley ® Healthy Chips double chocolate cookies fat free
1¼ bars Health Valley ® Date Bakes Fat Free cereal bars (3.9 g fiber)
2:90 Fudge bars
1 bar-Klondike ® Slim-a-bear 98% fat free no sugar added premium fudge bar or 1 ice cream
sandwich (90 cal, 4 g fiber)
1 Healthy Choice ® Premium ice cream bar (80 cal, 4 g fiber)

In addition to safe starches, the dieter may take two portions of fruit per day. Table 5 is a list of the fruits the dieter can choose to include in his or her daily menu, in accordance with the inventive diet. In accordance with the invention, fresh or frozen fruit may be employed.

Dieters should make sure that frozen fruit used is fruit only, by checking to see that the ingredients list lists only fruit and no added juices, sugars, syrups, etc. The dieter may use fruits to make smoothies occasionally. However, juicing is not recommended since juice contains little or no fiber. Suitable portions of fruit are listed in Table 5. Dried fruit that contains added sugars or syrups in the ingredients list should be avoided. Fresh or frozen fruit is allowed. Frozen fruit should list only fruit in the ingredients without added juices, sugars or syrups.

TABLE 5
2:90 Fruits
The following amounts are equivalent to approximately 90-calories.
One fruit can be substituted for:

Asian pear, 1 medium (4 g fiber)
Apple, 1 large with skin (4 g fiber)
Applesauce, 7 oz unsweetened (3.6 g fiber)-the only ingredients
it should list are apples, water and ascorbic acid to maintain color.
The best applesauce provides about 2 grams of
fiber for every 50-calorie serving.
Apricots, fresh 6 whole (3.7 g fiber)
Apricots, dried 12 halves (3 g fiber)
Annona, ½ of a 3 inch fruit (4 g fiber)
Blackberries, 1⅛ cup (10 g fiber)
Blueberries, 1⅛ cup (3.5 g fiber)
Boysenberries, 1¼ cup (9 g fiber)
Banana, 1 medium 6″ (2.7 g fiber)
Cantaloupe, 1½ cups (2.4 g fiber)
Cherries, 18 fresh (3 g fiber)
Cranberries, fresh 1¾ cup (9 g fiber)
Currants, 1⅓ cups (6.9 g fiber)
Dates, 4 whole or pitted (2.8 g fiber)
Figs, 3 medium (3.6 g fiber)
*Grapes, 25 small (1.2 g fiber)
Guavas, 2 (9 g fiber)
Grapefruit, ¾ of a large fresh (3.4 g fiber)
Honeydew melon, 1½ cups (2 g fiber)
Kiwi, 1½ (4 g fiber)
Kumquats without refuse, 7 (8.3 g fiber)
Litchis, ¾ cup (2 g fiber)
Mango, ¾ cup (2.5 g fiber)
Mamey sapote, 1 cup (6 g fiber)
Mulberries, 1½ cups (3.6 g fiber)
Nectarine, 1 large (3 g fiber)
Orange, 1 large (4.3 g fiber)
Passion fruit, 3.5 oz (10 g fiber)
Peach, 1 large, fresh (3 g fiber)
Pear, fresh 1 large (4.8 g fiber)
Plums, 3 small, 2″ diameter (2.7 g fiber)
Plums, dried 4½
Prunes, 7 breakfast prunes (2 g fiber)
Papaya, 1½ cups (4 g fiber)
Pineapple, 1⅛ cup (2.7 g fiber)
Raspberries, 1½ cups (11 g fiber)
*Raisins, 3 Tbsp (1.2 g fiber)
Star fruit, 2 cups (6 g fiber)
Strawberries, 1¾ c whole (5.1 g fiber)
Tangerines, 3 small (4.5 g fiber)
Tamarinds, 18 (2 g fiber)
*Watermelon cubes, 2 cups (1.2 g fiber)
*= These foods do not meet 2:90 rule.

The breakfast menu for weeks one and two for the female dieter also includes a portion of milk or yogurt. Table 6 is a list of milk and yogurt portions which the dieter can choose to include in his or her daily menu.

TABLE 6
Milk/Yogurt
The following amounts are equivalent to approximately 90-calories.
One milk/yogurt can be substituted for:

1 cup Fat free (skim) milk
1 cup 1% milk
1 cup light soymilk
¾ cup plain, vanilla, or unsweetened soy milk
¾ cup low fat plain Kefir
1 cup non-fat plain Kefir
1 cup Fat free buttermilk
¾ cup of low fat buttermilk
½ cup Evaporated fat free milk
⅓ cup (dry) Fat free dry milk
¾ cup plain nonfat yogurt
1 container of any flavored 2:90 yogurt (providing at least 2 grams
of fiber for every 90 calories)

The dieter has more flexibility in selecting vegetables to complete his or her daily menu. The dieter should not overcook vegetables to preserve nutrient values.

Juicing is not recommended as it separates out the fiber and removes it from the fruit or vegetable. In accordance with the inventive diet, one portion of vegetable is defined as a 90 calorie serving and 7.5 grams of fiber.

The fat portion in the female dieter's first and second week breakfast option, for example, can be two teaspoons of any of the oil options listed in Table 7. Alternatives are provided in Table 7. The female dieter may have three servings of fats per day. The male dieter may have four servings of fat per day. The fat servings may be used to fry a food item such as an egg white, in a salad dressing with an unrestricted amount of vinegar, as a flavor enhancer in a portion of rice, or shared equally or unequally between any or all of these applications.

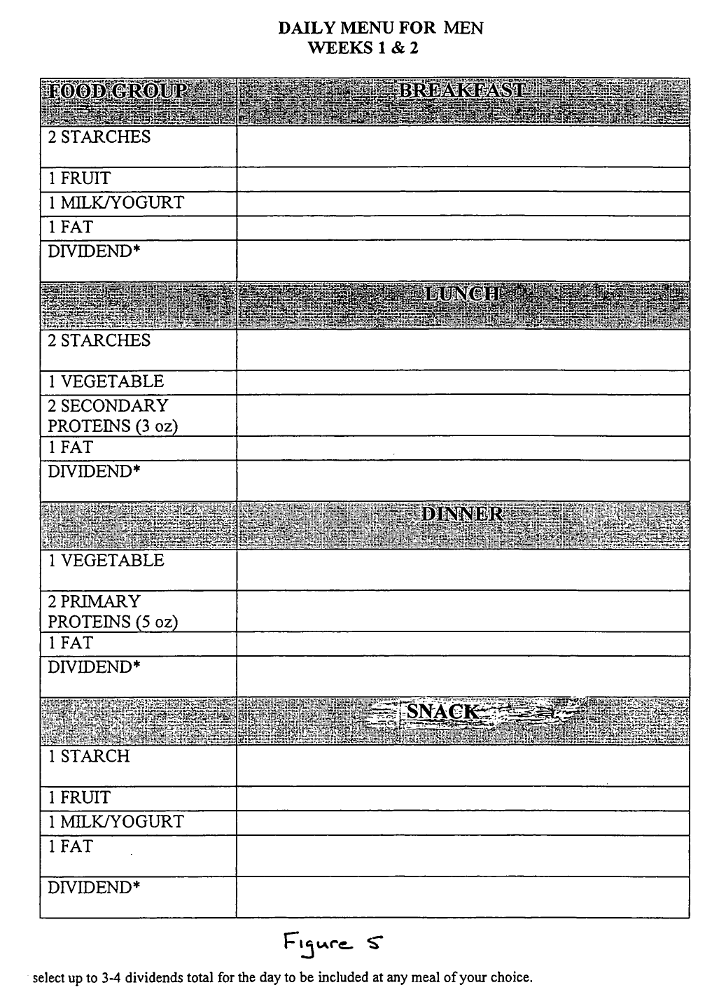

Table 7 is a list of fat portions the dieter can select to include in his or her daily menu, and like the other tables contained herein, apply to the diet elements detailed in the lists represented by the forms of FIGS. 1-8, contained herein. The distribution of the food groups within the meal plans shown in FIGS. 1-4 for women and FIGS. 5-8 for men may be followed as shown or may be altered by the dieter using equivalent foods. As long as the daily consumption of foods results in the recommended percentages of carbohydrates, proteins and fats, and the 2:90 Rule is followed, the dieter or the dieter's counselors may vary the menus and meal plans to add variety and suit the needs of the individual dieter.

The best oils are fresh cold pressed, and mechanically processed in opaque glass containers. Flax/linseed oil should be added to food after cooking. This oil can be used in salad dressings, on popcorn, in yogurt, on cereal (hot or cold), and in dips. However, flaxseed may increase bleeding time, so a dieter should consult his or her medical professional if he or she is taking blood-thinning medications.

TABLE 7
Fats

The following amounts are equivalent to approximately 90-calories.
One fat can be substituted for:
2 teaspoons of:
Wheat germ oil
Canola oil
Olive oil
Peanut oil
Avocado oil
Toasted pumpkin seed oil
Hazelnut oil
Sesame oil
Mac nut oil
Peanut oil
Walnut oil
Grape seed oil
Corn oil
Safflower oil
Soybean oil
Sunflower oil
1 Tablespoon of:
Natural peanut butter
Cashew butter
Almond butter
Almond paste
Soy nut butter
2 Tablespoons of:
Pumpkin seeds
Sunflower seeds
Flax seeds (5 g fiber)
All Natural Nayonaise soy based sandwich spread
Sesame seeds
Light margarine (with no trans fat)
Mayonnaise (reduced fat)
Reduced fat Miracle Whip salad dressing
4 Tablespoons of:
Avocado (2.6 g fiber)
Reduced fat salad dressing
Other Fats:
4 tsp of prepared pesto
16 large Ripe black olives (high sodium)
20 green olives (high sodium)
10 Kalamata olives (high sodium)
12 whole almonds (1.2 g fiber)
12 whole cashews (0.5 g fiber)
¼ cup Pistachios with shells
⅛ cup shelled hazelnuts (1.5 g fiber)
12 mixed nuts
20 Peanuts
8 Pecan halves (1.2 g fiber)
8 Walnut halves (1 g fiber)
⅛ cup black walnuts (1 g fiber)
1¾ Tbsp soy nuts
4 tsp Tahini paste
II. Any of the following fats count as one serving.
2 Tbsp of:
2 Tbs Take Control ® light vegetable oil spread
2 Tbs Benecol ® Light
2 Tbs Fleischmann's ® Light margarine
2 Tbs Country Crock ® light
2 Tbs Country Crock ® plus yogurt
2 Tbs I Can't Believe It's Not Butter ® light
2 Tbs Brummel & Brown ® spread made with yogurt
2 Tbs Smart Balance ® Light Buttery Spread
2 Tbs Smart Balance ® Omega Plus Light Buttery Spread
2 Tbs Hellmann's ® Reduced Fat Mayo
2 Tbs Hellmann's ® light Mayo
2 Tbs Kraft ® light mayo
2 Tbs Reduced fat Miracle Whip ® salad dressing

SALAD DRESSINGS

One serving of fat equals:
4 Tbsp Kraft ® Just 2 Good (70 cal, 4 g fat)
4 Tbsp Maple Grove Farms Fat Free (80 cal, 0 fat)
2 Tbsp Publix ® Lite Ranch (70 cal, 4.5 g fat)
2 Tbsp. Ken's Steak House ® Raspberry Vinaigrette (80 cal, 6 g fat)
4 Tbsp Newman's Own ® Lite Balsamic Vinaigrette (100 cal, 8.5 g fat)
4 Tbsp Balsamic Vinegar (20 cal, 0 fat)- FREE FOOD
4 Tbsp. Red Wine Vinegar (0 cal, 0 fat) - FREE FOOD

The inventive diet shows the dieter the key to a successful weight loss strategy by showing the dieter how to create meals that meets his or her own needs. In addition, the dieter can include up to four “dividends” for the day. The inventive diet encourages men and women to additionally select up to four dividend foods selected from foods and beverages substantially devoid of caloric value, foods and beverages in quantities sufficiently small that such foods and beverages are substantially devoid of caloric value, fat free condiments and combinations thereof. Table 8 is a list of dividends the dieter can choose to include in the daily meal plan.

These foods have a great diversity and can be used to keep interest in the regimen high. Condiments, some desserts and treats, and so forth can be included as dividends.

The concept of the dividend is that the overall healthful nature of the other items in a given meal plan allows the introduction into the diet of elements that are traditionally viewed as inconsistent with dieting, but which are earned with the overall quality of the meal. Moreover, moderate portions of such are satisfying because of the filling and varied nature of foundation elements of the inventive diet.

TABLE 8
Dividends
3-4 dividends may be selected each day from the following list.

| EVERYDAY FOODS | THE HEALTHIER ALTERNATIVE |
|---|---|
| 16 oz Propel ® Fitness Water | |
| 4 oz of vegetable or tomato juice | |
| 2 Tbsp 2% milk or fat free evaporated milk | |
| (can be used as cream in coffee) | |
| 2 oz skim milk | |
| 1.5 oz 1% milk | |
| 4 tsp dry nonfat milk | |
| 1 Tbsp nondairy liquid creamers | 1 Tbsp organic half & half or |
| | 1 Tbsp soymilk creamer |
| 2 tsp nondairy powdered creamers | |
| 2 tsp Postum ® instant hot beverage | |
| 1 teaspoon white, granulated sugar (not for | 1 teaspoon brown sugar (not for diabetics) |
| diabetics) | |
| 1 sugar cube (not for diabetics) | |
| 2 tsp powdered sugar (not for diabetics) | |
| 1 teaspoon honey (not for diabetics) | |
| 3 pieces of The Laughing Cow ® light gourmet | |
| cheese bites | |
| 1 Tbsp Cream cheese, fat free | |
| 2 tsp grated Parmesan cheese | 2 tsp low fat veggy Parmesan made from soy |
| 1 egg white | |
| 1 Tbsp Mayonnaise fat free | |
| 1 tsp reduced fat mayo | |
| 2 Tbsp Margarine, fat free | |
| 1 tsp reduced fat margarine | |
| 1 tsp light butter | |
| Nonstick cooking spray (a 3-second spray) | |
| 2 tsp sour cream | |
| 1 Tbsp light sour cream | |
| 2 Tbsp fat free sour cream | |
| 1 Tbsp Non-fat Miracle Whip ® | |
| 1 tsp reduced fat Miracle Whip ® | 1 Tbsp Vegenaise |
| 1 Tbsp Salad dressing fat free | 2 Tbsp Up Country Organics of |
| | Vermont organic balsamic vinaigrette |
| | naturally fat free or |
| | 1 Tbsp Annies Naturals ® organic no |
| | fat yogurt dressing with dill |
| 7 classic-cut croutons | |
| or 3 restaurant-style croutons | |
| 3 tsp hummus | |
| 3 Tbsp Salsa | |
| 2 Tbsp Sauerkraut | |
| 1 Tbsp Worcestershire sauce | |
| 2 Tbsp Whipped topping, light or nonfat | 2 Tbsp Natural By Nature ® Whipped cream |
| 2 Hard candy, sugarless | |
| 1 sugar-free Popsicle | 1 pop All natural fruit juice freezers no sugar |
| | added |
| 2 Tbsp sugar-free jam or jelly | |
| 1 Tbsp low sugar Jam or jelly | 2 tsp organic no sugar added fruit |
| | spread or |
| | 2 tsp Organic spreadable fruit |
| | (containing no more than 7 grams of |
| | total carbohydrates per 1 Tbsp |
| | serving) or |
| | 2 tsp organic juice sweetened apple |
| | butter or |
| | 1 tsp organic dried fig spread or |
| | 1 tsp organic tart cherry butter |
| 2 Tbsp Syrup, sugar free | 2 tsp Organic raspberry or French vanilla syrup |
| 2 Sugar free fat free meringues | |
| 1 Tbsp Cocoa powder, unsweetened | |
| 1 Tbsp Ketchup (15 cal 4 g sugar sodium | 1 Tbsp organic ketchup |
| 190 mg) | |
| 1 Tbsp of relish | |
| 2 tsp wheat germ (original toasted) | |
| 2 Tbsp toasted wheat bran (3.5 g fiber) | |
| 5 cocktail olives | |
| 1 Tbsp Taco sauce | |
| 1 Tbsp chili sauce | |
| 1 tsp garlic chili pepper sauce | |
| 2 tsp Salad Toppings | |
| 1 Tbsp Seafood sauce calorie free, sugar-free | |
| (high sodium) | |
| 1 Tbsp steak sauce (high sodium) | |
| 1 Tbsp barbecue sauce (should contain no fat | |
| and about 15 calories per Tablespoon) | |
| 2 tsp peanut sauce | |
| ¼ cup canned pumpkin (20 cal, 2.5 g fiber) | |
| 1 Tbsp tomato paste (15 cal and 10 mg | |
| sodium) | |
| ¼ cup tomato sauce (20 cal and 280 mg | ¼ cup No salt added tomato sauce |
| sodium) | |
| ¼ cup canned tomatoes | |
| 2 Tbsp Cooking wine | |
| 2 Tbsp Capers (high sodium) | |
| 1 Tbsp Soy sauce, lite (high sodium) | |
| 1 cup prepared (using ½ cube) Bouillon, | 1 cup prepared of natural chicken, vegetable |
| broth, low sodium (high in potassium) or | or beef stock Kitchen Basics ® |
| 1 cup prepared (using ½ cube) Bouillon, | |
| broth, consommé (very high sodium) | |
| 1 tsp Fish sauce (high sodium) | |
| 1 Tbsp stir fry sauce (high sodium) | |
| 1 Tbsp soy ginger sauce (high sodium) | |
| 1 Tbsp Teriyaki sauce (should contain about 15 | |
| calories per Tablespoon) high sodium | |
| 1 Tbsp Hoisin sauce (high sodium) | |
| | 2 sheets Sushi Nori toasted sea vegetable |
| | 1 Tbsp natural sweet relish |
| | 1 tsp Organic Mellow red miso soybean paste |
| | 2 tsp organic hummus |
| | 2 tsp organic babaghannouj |
| 3 fish anchovy fillets (high sodium) | |
| 1½ large Pickles, dill (high sodium) | |
| Pepperoncini (3 peppers) | |
| 3 Tomato slices | |
| Peppers, 4 rings | |
| ½ cup salad greens | |
| 1 roasted pepper, from jar | |
| ¼ cup raw onions | |
| 1 cup celery | |
| 10 Cucumber slices | |
| 1½ tsp minute tapioca | |
| 2 tsp coconut flakes | |
| 2 tsp marshmallow fluff | |
| 1 tsp semi-sweet chocolate (chunks or mini- | |
| morsels) | |
| 2 tsp whole ground flaxseed meal (1.3 g fiber) | |
| 2 tsp cornstarch | |
| 2 tsp corn meal | |
| 2 tsp flour (whole wheat, bread flour, self- | |
| rising, all-purpose, cake flour) | |

Those dividend foods that tend to be high in sodium and potassium are identified in the Table above for those individuals with specific conditions that require that they monitor their salt intake closely. In addition, those monitoring their sodium intake should check food labels for a product's sodium content, even in those products that claim to contain no MSG.

As stated above, each of lunch and dinner for the female dieter may include one-half of a vegetable portion and for the male dieter may include one full vegetable portion. Table 9 provides exemplary vegetables that meet the 2:90 Rule.

TABLE 9
2:90 Vegetables
To make sure that the dieter consumes the right amount of fiber for the day;
one serving of vegetable should include 3 selections from the following list

| | FIBER | | |
|---|---|---|---|
| | CONTENT (in | | FIBER CONTENT |
| | grams per 30 | COOKED | (in grams per 30 |
| RAW VEGETABLES | calorie serving) | VEGETABLES | calorie serving) |
| 3 cups Endive, | 4.8 | 1 cup turnip | 5 |
| chopped | | greens, chopped | |
| ¾ cup yam bean | 4.5 | 1½ cups mustard | 4.2 |
| (jicama) | | greens | |
| 1 cup Escarole, | 4 | 1½ cups bok choy | 4 |
| chopped | | | |
| 1-cup broccoli sprouts | 4 | 1 cup cabbage, | 3.6 |
| | | shredded | |
| 2 cups celery | 3.8 | ½ medium | 3.5 |
| | | artichoke | |
| 1 cup green beans | 3.7 | 1 cup cauliflower | 3.4 |
| 2 cups turnip greens, | 3.6 | 1 cup turnip cubes | 3.2 |
| chopped | | | |
| 3 cups lettuce, Cos or | 3.6 | ¾ cup Chayote, | 3.2 |
| Romaine, shredded | | pieces | |
| 36 leaves Celtuce, | 3.6 | ½ cup Brussels | 3.2 |
| | | sprouts | |
| 2¼ cups grape leaves | 3 | ½ cup okra, sliced | 3 |
| ¾ cup kohlrabi | 3 | ¾ cup spinach | 3 |
| 2 cups Chives, | 3 | ¾ cup Italian beans | 3 |
| chopped | | | |
| ¾ cup bamboo shoots | 3 | ¾ cup Wax beans | 3 |
| 1½ cups radishes | 2.9 | ¾ cup broccoli | 3 |
| 4-cups lettuce, butter | 2.8 | ⅓ cup Artichoke | 2.9 |
| head Boston & Bibb | | hearts | |
| types | | | |
| 1-cup fennel | 2.7 | ½ cup collard | 2.7 |
| | | greens | |
| 1 cup spring onions or | 2.6 | 1 cup Celery, diced | 2.4 |
| scallions, chopped | | | |
| 1 cup spring onions or | 2.6 | 1½ cups zucchini | 2.1 |
| scallions, chopped | | | |
| 1-cup cauliflower | 2.5 | 2 cups Bamboo | 2 |
| | | shoots | |
| ¾ cup peppers, | 2.4 | ½ cup carrots | 2 |
| chopped | | | |
| 4-cups lettuce, iceberg | 2.4 | ½ cup mushrooms | 2 |
| 3-cups lettuce loose | 2.4 | ⅔ cup green | 2 |
| leaf | | beans or snap | |
| | | yellow beans | |
| 3 cups alfalfa sprouts | 2.4 | ¾ cup eggplant, | 2 |
| | | cooked | |
| 1-cup broccoli | 2.4 | ¾ cup kale | 2 |
| ½ cup snow peas or | 2.3 | ¾ cup slices | 2 |
| sugar snap peas | | summer squash, all | |
| | | varieties | |
| ⅕ of bunch broccoli | 2.3 | 3 cups Swiss chard | 1.8 |
| rabe | | | |
| 1-cup tomatoes | 2.2 | 1½ cup Pumpkin | 1.8 |
| | | flowers | |
| 1 cup Rhubarb, diced | 2.2 | 1½ cups pumpkin | 1.8 |
| | | flowers | |
| 1½ cups spinach, | 2.1 | ⅔ cup asparagus, | 1.8 |
| chopped | | about 6 spears | |
| 1½ cups cabbage, | 2.1 | ¾ cup Peppers, | 1.5 |
| shredded | | chopped | |
| ½ cup carrots | 2 | | |
| 1 cup onion sprouts | 2 | ½ cup slices beets | 1 |
| 4½ cups arugula | 1.8 | ½ cup kohlrabi | 1 |
| 1 medium red tomato, | 1.6 | | |
| raw | | | |
| 2 cups mushrooms | 1.6 | ½ cup Kohlrabi, | 1 |
| | | slices (12 cal, 0.5 g | |
| | | fiber) | |
| 1 cup slices summer | 1.6 | ¾ cup leeks, | 0.9 |
| squash, all varieties | | chopped | |
| 1½ cup cucumbers, | 1.5 | ¼ cup Onions, | 0.8 |
| sliced | | chopped | |
| ½ cup raw onions, | 1.4 | | |
| chopped | | | |
| 6 cups watercress | 1.2 | | |
| 3-cups radicchio | 1.2 | | |
| 1½ cup slices | 1.2 | | |
| zucchini | | | |
| 8 broccolini stalks | 1 | | |
| ¼-cup water | 1 | | |
| chestnuts, Chinese | | | |
| Matai | | | |
| ½ cup leeks, chopped | 0.8 | | |
| 1 cup bean sprouts | 0.6 | | |

Lunch for the female dieter during the first two weeks also includes one secondary protein portion, which may be selected from the list of secondary protein portions shown in Table 10. The dieter can select any of these to include in the daily menu in accordance with the figures for any phase of the men's and women's diet.

TABLE 10
Secondary Proteins
The following amounts are equivalent to approximately 90-calories.
One secondary protein is equivalent to:

1.5 ounces cooked of:
Lamb (Roast, chop, leg)
Veal (Lean chop, roast)
Chicken or turkey (dark meat, skinless)
1 chicken drumstick no skin = 1.5 ounces cooked meat
1 chicken leg no skin = 3.5 ounces cooked meat
1 small chicken leg = 2 ounces
1 chicken thigh no skin = 2 ounces cooked meat
1 chicken wing no skin = 1 ounce cooked meat
Herring (uncreamed or smoked)
Fresh (wild Atlantic) or canned salmon
Tuna canned in oil, drained
Catfish
Mackerel
Pork (lean pork, fresh ham, canned, cured or boiled ham, tenderloin,
center loin chop)
Beef: USDA Select or Choice grades of lean beef with visible fat
trimmed, best if grass fed beef.
Round, sirloin and flank steak
Tenderloin (Filet Mignon)
Roast (rib, rump, chuck)
T-bone steak
Porterhouse steak
Cubed steak
Ground round
Domestic duck (skinless, well-drained of fat)
Goose (skinless, well-drained of fat)
Rabbit
Any cheese with more than 1 but no more than 3 grams of fat per ounce
Deli meats with more than 1 but no more than 3 grams of fat per ounce
Natural smoked light tuna slices with ginger in sunflower oil
Other:
3 oz (firm, soft, lite firm) organic unflavored tofu
6 oz silken organic tofu
1¾ oz tempeh
2 oz Edamame (soybeans)
1 Veggie Burger patty (recommend Gardenburger ® veggie medley
or Morningstar Farms ® garden veggie patties)
2¼ oz hot dogs with more than 1 but no more than 3 grams
of fat per ounce
4 slices Canadian bacon (high sodium)
6 slices Schneider's Genuine Canadian bacon (high sodium)
3 medium canned sardines
9 medium oysters
1 mozzarella string cheese with no more than 4 grams of total
fat per string
3.5 Tbsp grated Parmesan cheese
1 oz reduced fat feta cheese with no more than 4 grams of total fat
per ounce
1 whole egg** + 1 egg white (**Limit whole eggs to 3 per week,
recommend organic cage free eggs).

As shown in the figures, each meal for the male and female dieter also may include a portion of a dividend which may be selected from the dividends shown in Table 8.

Dinner for the female dieter during the first two weeks may include two portions of a primary protein, as can be seen with reference to FIG. 1. The difference between primary and secondary proteins is the fat content. Secondary proteins are those that contain more fat than the primary proteins. Hence, the portion size of a secondary protein is smaller than that of a primary protein.

Primary proteins may be selected from the examples set forth in Table 11.

TABLE 11
Primary Proteins
The following amounts are equivalent to approximately 90-calories.
One primary protein is equivalent to:

2.5 ounces cooked of:
Chicken breast (no skin) - one chicken breast, skinless is approximately
3 ounces.
Turkey breast (no skin)
Cornish hen (no skin)
Goat, roasted
Fresh or frozen cod
Haddock
Flounder
Tilapia
Mahi-Mahi
Swordfish
Grouper
Snapper
Whitefish
Halibut
Trout, rainbow wild
Tuna fresh
Canned tuna in water (may use low sodium if desired)
Clams
Crab (contains more cholesterol)
Lobster (contains more cholesterol)
Scallops
Shrimp (contains more cholesterol)
Duck or pheasant (skinless)
Venison
Buffalo
Ostrich
Fat free cheese
Deli meats with 1 gram or less fat per oz
Hot dogs with 1 gram or less fat per ounce (may be high sodium)
Sausage with 1 gram or less fat per ounce (may be high sodium)
Vegetarian stir-fry strips seasoned wheat gluten
½ cup of:
Cooked beans (garbanzo, black, pinto, lima beans, kidney, cannelloni,
white, split, lentils, black-eyed) vegetarian style.
Best if made from scratch. Canned beans contain much more sodium.
If no other alternative than canned is available, dieters are encouraged
to select one that contains under 300 mg sodium per serving.
Imitation crabmeat (high sodium)
Fat free or low fat cottage cheese containing up to 1% milk fat
Fat free Ricotta cheese
Other:
5 egg whites (or approximately ⅔ cup of any egg white product)
2 oz Natural wild caught skinless & boneless sardines in water
3.5 oz natural smoked light tuna slices in water
4 slices extra lean turkey bacon 95% fat free (high sodium)

As is detailed in FIG. 1, dinner for the female dieter during the first two weeks of the diet is completed by a fat, a half portion of a vegetable and a dividend, selected as detailed above.

As detailed in the chart/form of FIG. 1, the initial daily diet regimen for the female dieter in the first two weeks is completed by a healthy dessert or snack taking the form of a portion of a starch, a portion of a fruit, a portion of a milk or yogurt, a portion of a fat, and a portion of a dividend. Adjustments to the number of servings may be made if the individual is losing more or less than the recommended two pounds per week.

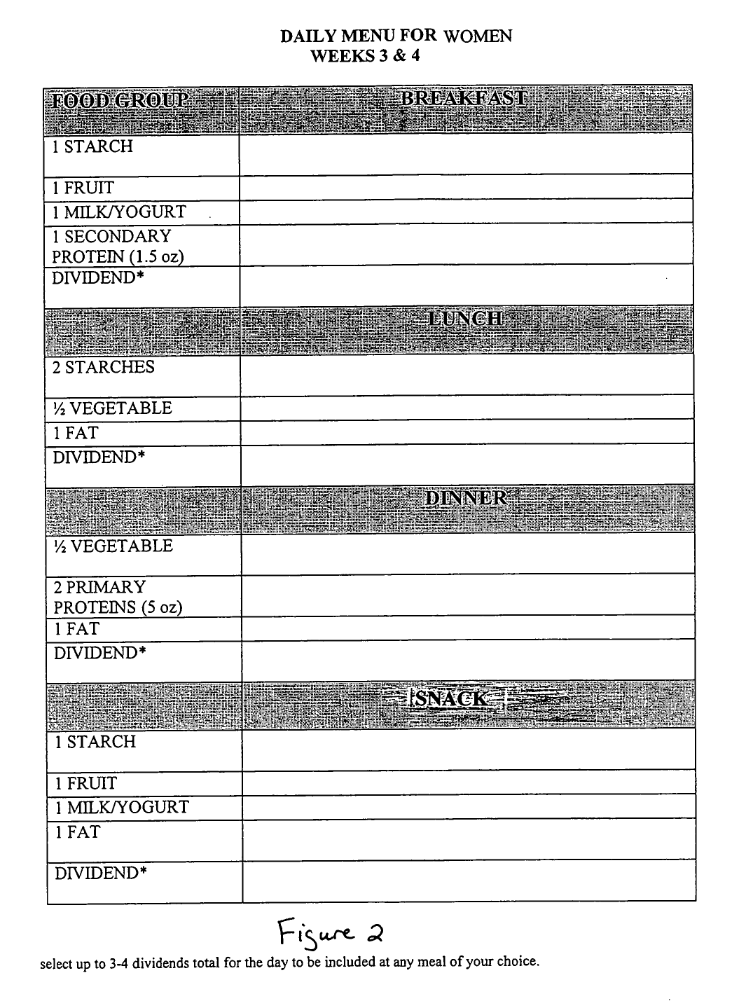

During the third and fourth weeks of the inventive diet, the female dieter follows the regime outlined in FIG. 2. The various categories of food options, such as primary and secondary proteins, fats, fruits and so forth are selected in accordance with the information outlined in the form of a chart with blanks in FIG. 2, using the tables detailed herein.

The dieter or the dieter's physician, nutritionist, counselor or other health care provider can follow the recipes provided herein, or can create a wide variety of meals based on the herein recommended food groups with reference to the equivalence amounts listed in the Tables provided herein. The recipes are interchangeable.

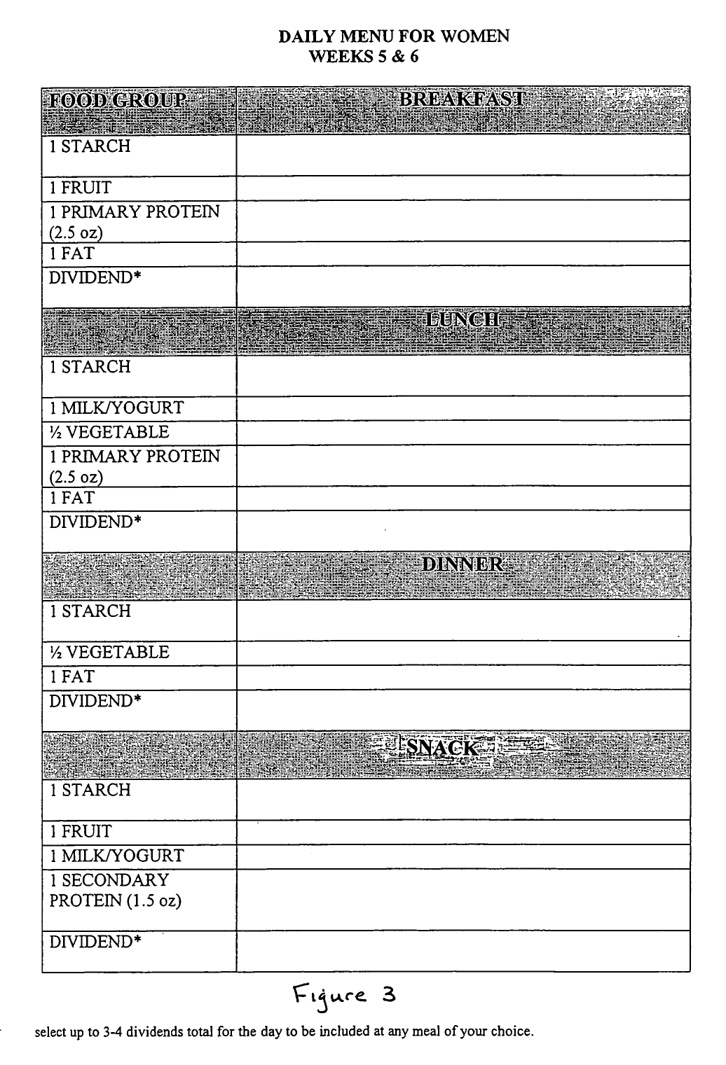

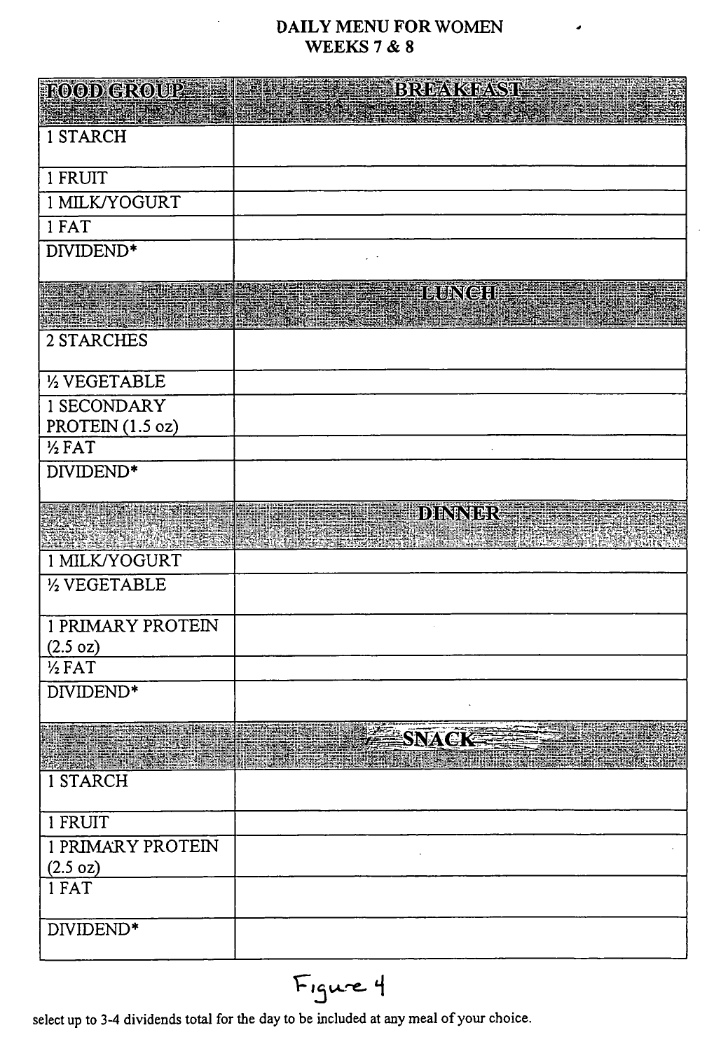

During the first eight weeks of the inventive diet, the female dieter may follow the regime outlined in FIGS. 1-4, using the tables detailed herein. In that event, during the first two weeks of the inventive diet, the female dieter would follow the regimen outlined in FIG. 1. During the third and fourth weeks of the inventive diet, the female dieter would follow the regimen outlined in FIG. 2, using the tables detailed herein. During the fifth and sixth weeks of the inventive diet, the female dieter would follow the regimen outlined in FIG. 3, again using the tables detailed herein. During the seventh and eighth weeks of the inventive diet, the female dieter would follow the regimen outlined in FIG. 4, again using the tables detailed herein. The various categories of food options, such as starches, primary and secondary proteins, fats, fruits and so forth may be selected in accordance with the information outlined in the form of a chart with blanks in FIGS. 1-4. The dieter should follow the same meals on different week patterns until the goal weight is reached. After week eight, the menus shown in FIGS. 1-4 may be reused by the female dieter or she by then would be used to the options and equivalents, so that she may plan her own meals using the principles and guidelines set forth herein. The total daily caloric intake for women may initially include four servings of starch, two servings of fruit, two servings of milk or yogurt products, three servings of fats, two servings of primary proteins, one serving of secondary proteins and one serving of vegetables.

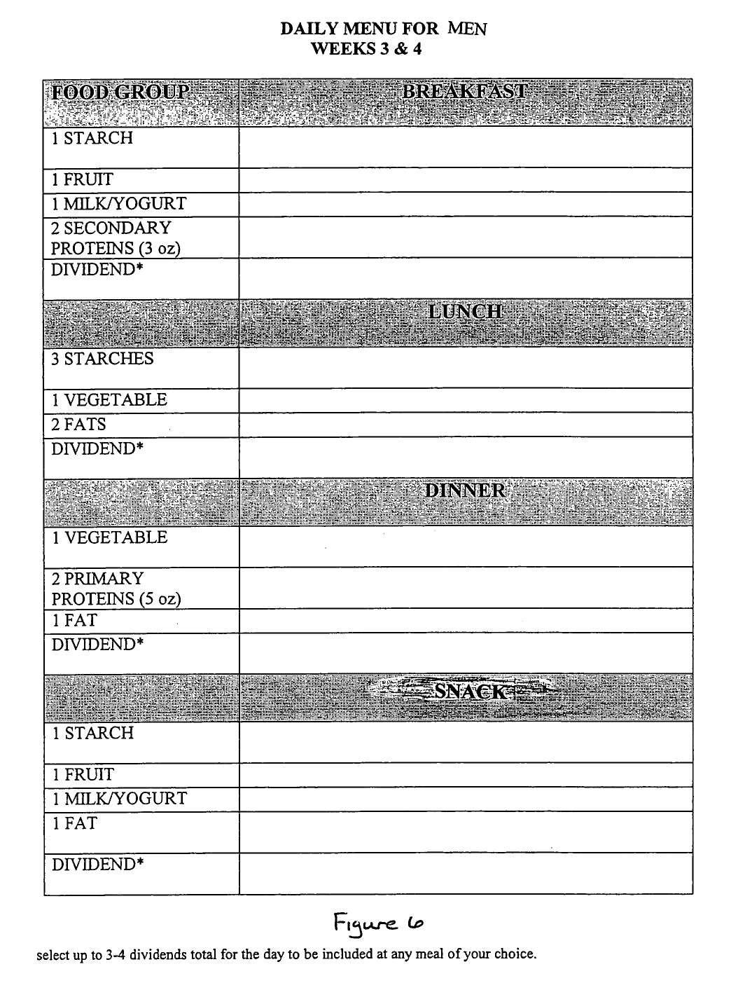

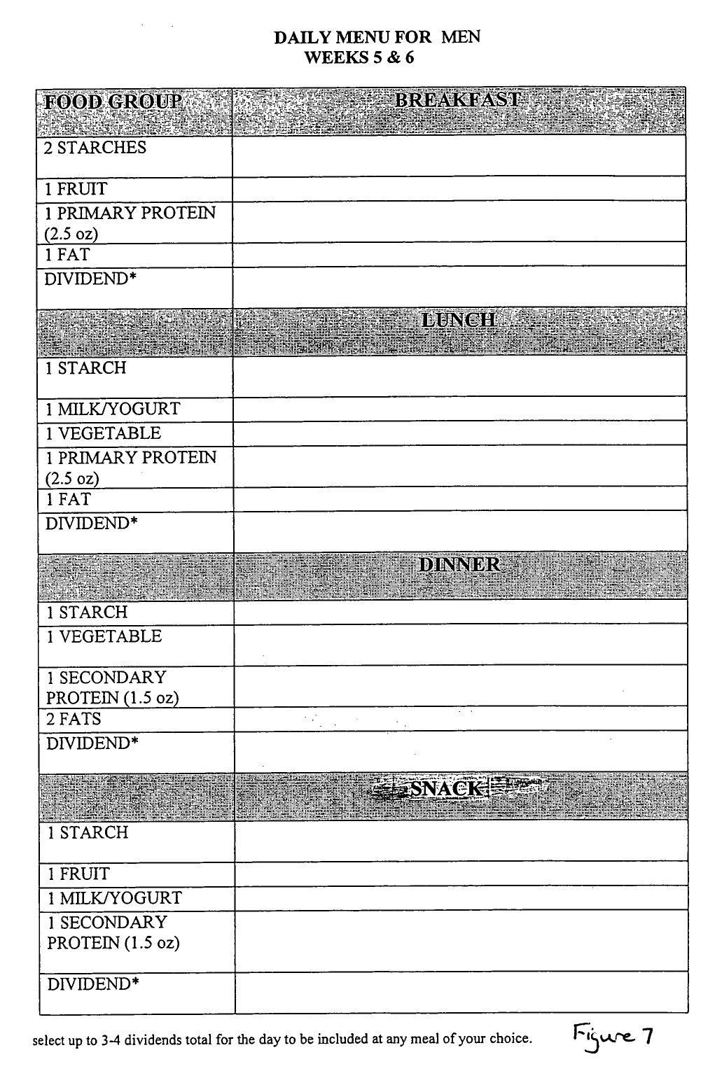

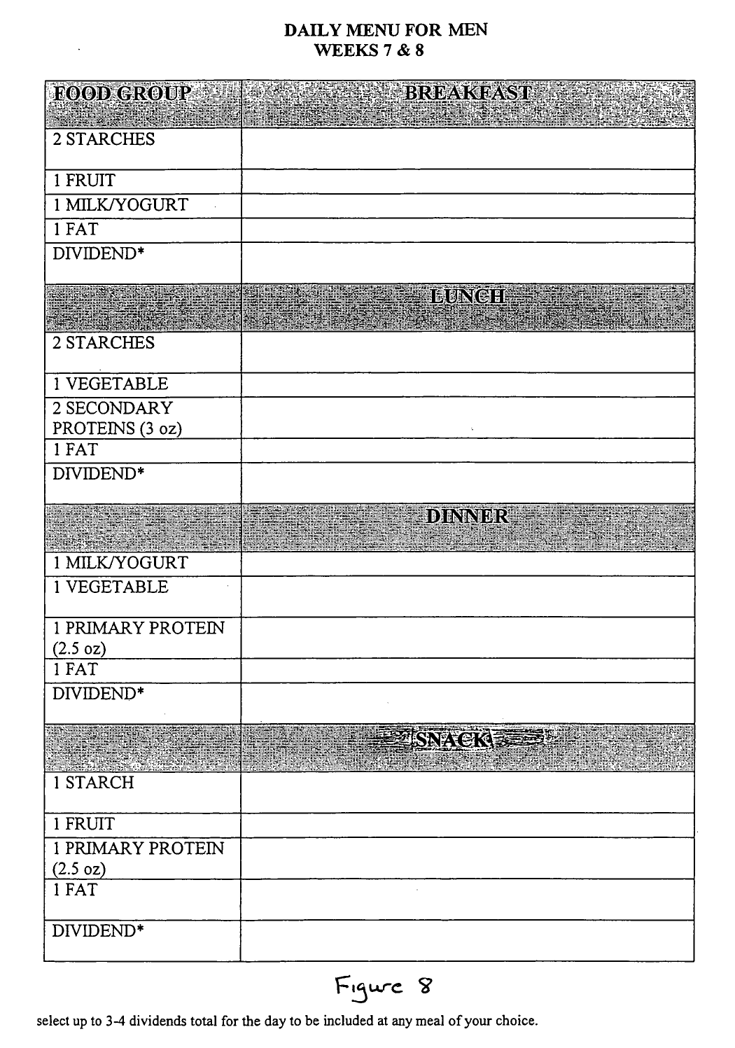

Similarly, during the first two weeks of the inventive diet, the male dieter choosing to use the charts provided herein would follow the regimen outlined in FIG. 5. During the third and fourth weeks of the inventive diet, the male dieter would follow the regimen outlined in FIG. 6, using the tables detailed herein. During the fifth and sixth weeks of the inventive diet, the male dieter would follow the regimen outlined in FIG. 7, using the tables detailed herein. During the seventh and eighth weeks of the inventive diet, the male dieter would follow the regimen outlined in FIG. 8, using the tables detailed herein. As with the female dieter, the various categories of food options, such as starches, primary and secondary proteins, fats, fruits and so forth are selected in accordance with the information outlined in the form of a chart with blanks in FIGS. 5-8, using the tables detailed herein. The total daily caloric intake for men may initially include five servings of starch, two servings of fruit, two servings of milk or yogurt products, four servings of fats, two servings of primary proteins, two servings of secondary proteins and two servings of vegetables.

The inventive diet encourages men and women to additionally select up to four dividend foods selected from foods and beverages substantially devoid of caloric value, foods and beverages in quantities sufficiently small that such foods and beverages are substantially devoid of caloric value, fat free condiments and combinations thereof, as described more fully below.

Adjustments to the number of servings may be made if the individual is losing more or less than the recommended two pounds per week. For example, if a man starting the diet at a daily caloric intake of 1800 calories or a woman starting the diet at a daily caloric intake of 1400 calories is losing more than two pounds per week, he or she may add an additional serving per day during the following week. If the dieter still loses more than two pounds per week, he or she may add a second additional serving to the daily total food intake and so on until the weight loss stabilizes at two pounds per week. After the dieter reaches the desired weight objective, one or more servings of food may be added to the daily total until the weight stays steady at or about the desired weight to maintain the desired weight.

Dieters, in accordance with the invention are directed to minimize caffeine intake, preferably keeping it below 300 mg, and most preferably below 250 mg per day. Table 12 provides information regarding daily caffeine intake.

TABLE 12
Caffeine content of some foods

| | | |
|---|---|---|
| Coffee, brewed, 1 cup | 135 | mg |
| Coffee, brewed, decaffeinated | 2 | mg |
| Coffee, drip, 1-cup | 150 | mg |
| Coffee, drip, decaffeinated | 5 | mg |
| Coffee, instant | 75 | mg |
| Coffee, instant, decaffeinated | 3 | mg |
| Coffee, espresso (2 oz) | 100 | mg |
| Coffee, cappuccino (single) | 100 | mg |
| Coffee, latte (single) | 100 | mg |
| Coffee, mocha (single) | 100 | mg |
| Coffee substitute, cereal grain | | |
| Beverage or herbal tea | 0 | mg |
| Black tea 8 oz | 50 | mg |
| Green tea 8 oz | 40 | mg |
| Oolong tea or iced tea 8 oz | 45 | mg |
| Cola 12 oz can | 30-45 | mg |

The inventive diet offers professional menu suggestions that may be followed using very simple and quick recipes. The menus provide a lot of variety, an important factor in promoting adherence to a weight-loss plan. The inventive diet portions cover a variety of ethnic cuisines, including American, Italian, Mexican, Spanish, etc. When the dieter eats out, he or she can choose a dish that closely resembles the recommended menu.

The inventive diet also indicates which free, or dividend, foods tend to be high in sodium and potassium, for those individuals with specific conditions, requiring that they monitor their intake closely. In addition, if the dieter monitors his or her sodium intake, make sure to check food label for the product's sodium content, even in those products that claim to contain no MSG.

Table 13 is a list of foods the dieter can enjoy liberally when observing the inventive diet. Some of these foods have no or minimal nutritive value, but some, for example water, are important to health. In the context of the inventive diet, they help to address psychological cravings, and impulses to eat based on nervousness, impulse or unsettled emotional or decisional states. Others add interest and flavor to foods which may have limited fat or sugar content.

TABLE 13
Free Foods
Enjoy the following foods liberally

| | THE HEALTHIER |
|---|---|
| EVERYDAY FOODS | ALTERNATIVE |
| Water | |
| Club soda | Natural Hansen ®'s |
| | club soda |
| Sparkling natural mineral or spring water | |
| 8 oz Seltzer sparkling mineral water (avoid | |
| those with sugar) | |
| Tonic water, sugar free | |
| Sugar free gelatin (1 a day is not counted) | |
| Sugar free gum (2 a day not counted) | |
| Sugar substitutes | Splenda ® |
| Decaf coffee (filtered, percolated, or instant) | Caffeine-free herbal coffee |
| Limit regular coffee to 1-2 cups per day | |
| Decaf teas | Herbal teas |
| Limit regular diet soda to 1-2 per day | |
| Unsweetened iced tea | |
| Other sugar free, diet drink mixes | |
| Horseradish | |
| Mustard | Organic mustard |
| Lemon juice or Limejuice | |
| Vinegar | |
| Flavoring extracts | |
| Mint sauce | |
| Herbs & Spices | |
| Tabasco ® or hot pepper sauce | |
| Baking soda | |
| Baking powder | |

To aid the dieter in weight management, various means of keeping track of food types and portions consumed may be used. For example, the forms illustrated in FIGS. 1-8 may be taken to supermarkets or restaurants and filled in as food purchases. Forms may take paper or electronic form, for example as files in an electronic organizer, notepad, notebook computer or hand-held communications or organizational device such as a Blackberry®, Palm Pilot® or similar device. The inventive diet may provide exemplary weekly shopping lists which may be provided with reference to corresponding weekly recipes. The recipes may also be provided in paper or electronic form and are interchangeable.

Other means of keeping track of the food types, amounts or portions consumed and exemplary suggested recipes include interactive software on a cd rom or hard drive, or a website, which may include useful advertising and links to information of interest to the dieter.

The rules and recommended food selections for the diet may be presented in a conventional book format or by electronic means, such as an electronic device or in a software product or on a website. The food tables contained herein together with the meal planning forms of FIGS. 1 through 8, the general guidelines respecting acceptable starches, the guidelines for proteins and the guidelines for fats, and the ratios of caloric intake for starches, proteins and fats may be obtained by the dieter or nutritionist, health care provider or counselor for advice to and use by the dieter by any of the foregoing means.

Compliance with the dietary regimen also requires a minimal amount of exercise. A recommended level of exercise would be enough to bum at least 1500, and preferably 2000 calories per week.

Time (in minutes) required to bum approximately 300 calories doing the activities shown in Table 14, based on the dieter's weight at the time of doing the exercise.

TABLE 14

| ACTIVITY | 125 lbs | 150 lbs | 175 lbs | 200 lbs |
|---|---|---|---|---|
| Aerobics, high impact | 39 | 32 | 28 | 24 |
| Vigorous full court | 25 | 21 | 18 | 15 |
| basketball | | | | |
| Bicycling: | — | — | — | — |
| 13 mph | 54 | 44 | 38 | 33 |
| 15 mph | 49 | 41 | 35 | 31 |
| 17 mph | 42 | 35 | 30 | 26 |
| 19 mph | 32 | 26 | 23 | 20 |
| 21 mph | 27 | 22 | 19 | 17 |
| 23 mph | 22 | 18 | 16 | 14 |
| 25 mph | 17 | 14 | 12 | 11 |
| Canoeing, moderate | 54 | 44 | 38 | 33 |
| pace in flat water | | | | |
| Cross-country skiing: | — | — | — | — |
| 8 mph | 23 | 19 | 16 | 14 |
| Golf (carrying your | 54 | 44 | 38 | 33 |
| clubs) | | | | |
| Handball | 31 | 26 | 22 | 19 |
| Horseback riding | 46 | 38 | 33 | 29 |
| (trot) | | | | |
| Rowing vigorously | 25 | 21 | 18 | 15 |
| Running: | — | — | — | — |
| 5 mph | 39 | 33 | 28 | 25 |
| 6 mph | 33 | 27 | 23 | 20 |
| 7.5 mph | 25 | 21 | 18 | 16 |
| 9 mph | 23 | 19 | 17 | 15 |
| 10 mph | 21 | 18 | 15 | 13 |
| 11 mph | 18 | 15 | 13 | 11 |
| Soccer (vigorous) | 25 | 21 | 18 | 15 |
| Studying | 3½ hours | 3 hours | 2½ hours | 2 hrs |
| | | | | 15 min |
| Swimming: | — | — | — | — |
| 20 yards per minute | 1 hr 15 min | 1 hr 3 min | 54 | 47 |
| 45 yards per minute | 41 | 34 | 29 | 26 |
| 50 yards per minute | 34 | 29 | 24 | 21 |
| Table tennis | 54 | 44 | 38 | 33 |
| (experienced) | | | | |
| Tennis (beginner) | 1 hr 15 min | 1 hr 15 min | 54 | 47 |
| Walking (fast pace): | — | — | — | — |
| 3.5 mph | 1 hr 8 min | 58 | 49 | 43 |
| 4.5 mph | 50 | 42 | 36 | 31 |
| Weight lifting: | — | — | — | — |
| Light to moderate | 1 hr 40 | 1 hr 23 | 1 hr 11 | 1 hr 3 |
| intensity | min | min | min | min |
| Vigorous intensity | 50 | 42 | 36 | 31 |
| Wheelchair basketball | 29 | 24 | 20 | 18 |
| Wheelchair (wheeling | 1 hr 19 | 1 hr 7 min | 57 | 50 |
| self) | min | | | |

### Dining Out Guide

Although the nutritional quality of some restaurant foods may be poor, the dieter may substitute the inventive diet treat once each week in order to free up to 4 food groups (a total of 360 calories). By freeing up these 4 food groups, the dieter can either replace them with other foods the same day or use them throughout the week as desired. Most of these items, even though may count as 1 or 2 food groups, may contain large amounts of fat, mainly saturated fat or simple carbohydrates such as sugars. The inventive diet is designed to help the dieter make the most nutritious daily food choices.

TABLE 15

| | | # Of food groups to |
|---|---|---|
| COMBINATION FOODS | SERVING SIZE | count as: |
| SANDWICHES/DELI | | |
| ITEMS | | |
| Turkey with mustard | 1 regular | 4 |
| sandwich | | |
| Roast beef with mustard | ½ | 3 |
| sandwich | | |
| Bacon & cheese grilled | ½ | 4 |
| chicken | | |
| Chicken salad | ½ | 3 |
| Egg salad | ½ | 3 |
| Ham with mustard | ½ | 3 |
| BLT sandwich | ½ | 3 |
| CHINESE | | |
| P.F. Chang's chicken with | ½ entree | 2 |
| black bean sauce | | |
| P.F. Chang's Moo Goo Gai | ½ entree | 3 |
| Pan | | |
| P.F. Chang's steamed | ½ entree | 3 |
| salmon | | |
| P.F. Chang's Ma Po tofu | ½ entree | 3 |
| P.F. Chang's garlic snap | ½ order | 2 |
| peas | | |
| Chow Mien without | 2 cups | 3 |
| noodles or rice | | |
| P.F. Chang's Cantonese | 1 entree | 3 |
| shrimp | | |
| P.F. Chang's Cantonese | 1 entrée | 3 |
| scallops | | |
| P.F. Chang's Pin rice | 1 order | 3 |
| noodle soup | | |
| P.F. Chang's mango | ½ entree | 3 |
| chicken | | |
| P.F. Chang's ginger chicken | ½ entree | 3 |
| with broccoli | | |
| P.F. Chang's shrimp with | ½ entree | 3 |
| lobster sauce | | |
| P.F. Chang's vegetable | ½ entree | 4 |
| chow fun | | |
| Stir-fried vegetables with | 2 cups | 4 |
| rice | | |
| P.F. Chang's Kung Pao | ½ entree | 4 |
| chicken | | |
| Oriental chicken salad with | ½ | 4 |
| dressing | | |
| Sesame prawn toasts | 1 piece | 1 |
| Hot and sour soup | 1 serving | 1 |
| Prawn wonton | Each | 1 |
| Prawn crackers | Each | 1 |
| Spare rib | Each | 2 |
| Crab and sweet corn soup | 1 serving | 2 |
| Chicken noodle soup | 1 serving | 2 |
| Chicken and sweet corn | 1 serving | 2 |
| soup | | |
| Crispy seaweed | 1 serving | 2 |
| Pancake roll | 1 serving | 3 |
| Chicken in lemon sauce | 1 serving | 3 |
| Chicken and pineapple | 1 serving | 3 |
| Beef in oyster sauce | 1 serving | 4 |
| Beef in yellow bean sauce | 1 serving | 4 |
| Beef with green peppers | 1 serving | 4 |
| and black bean sauce | | |
| Chicken and cashew nuts | 1 serving | 4 |
| Chicken chop suey | ½ serving | 3 |
| Sweet and sour chicken | ½ serving | 3 |
| SEAFOOD | | |
| Fried shrimp | ½ order | 3 |
| Shrimp with garlic sauce & | ¼ order | 3 |
| rice | | |
| Broiled seafood | 1 entree | 2 |
| ITALIAN FOOD | | |
| Olive Garden's Capellini | ½ entree | 3 |
| Pomodoro | | |
| Olive Garden's shrimp | ½ | 3 |
| primavera | | |
| Olive Garden's linguine alla | ½ entree | 3 |
| marinara | | |
| Olive Garden's chicken | ½ entree | 3 |
| Giardino | | |
| Melon with parma ham | 1 serving | 2 |
| Mozzarella and tomato | 1 serving | 2 |
| salad (no dressing) | | |
| Mixed fish salad | 1 serving | 3 |
| Bruschetta | 1 serving | 3 |
| Minestrone soup | 1 serving | 3 |
| Mushroom risotto | ½ serving | 3 |
| Tuna and bean salad | 1 serving | 3 |
| Spaghetti Bolognese | ½ serving | 4 |
| Garlic bread, 3 pieces | 1 serving | 3 |
| Spaghetti Arrabiata | ½ serving | 2 |
| Scampi Provencale | ½ serving | 3 |
| Cannelloni | ½ serving | 3 |
| Lasagna | ½ serving | 4 |
| Ravioli | ½ serving | 3 |
| Chicken risotto | ½ serving | 3 |
| Spaghetti Napoletana | ½ serving | 4 |
| Spaghetti marinara | ½ serving | 4 |
| Gelati | 1 serving | 2 |
| Cassata | 1 serving | 2 |
| Zabaglione | 1 serving | 2 |
| Tiramisu | ½ serving | 3 |
| MEXICAN FOOD | | |
| Refried beans | 1 side order | 4 |
| Tortilla chips and salsa | ½ serving | 4 |
| Tortilla chips and | ½ serving | 4 |
| guacamole | | |
| Panda Express orange | ½ | 3 |
| chicken with rice | | |
| Guacamole | ¼ cup | 1 |
| Sour cream | ¼ cup | 1 |
| Mexican rice | 1 side order | 3 |
| Quesadilla | ½ serving | 4 |
| Chicken burrito | ½ serving | 4 |
| Chicken enchilada | ½ serving | 4 |
| Chicken chimichanga | ½ serving | 4 |
| Beef burritos | ½ serving | 4 |
| Beef enchilada | ½ serving | 4 |
| JAPANESE | | |
| Sushi | 4 rolls | 3 |
| California roll | 9 pieces | 3 |
| M&M rolls (tuna & | 5 pieces | 1 |
| cucumber) | | |
| M&M roll (eel) | 12 pieces | 3 |
| Nigiri octopus rolls | 1 piece | 1 |
| Nigiri shrimp rolls | 1 piece | 1 |
| Nigiri (squid or tuna) | 1 piece | 1 |
| Nigiri Salmon rolls | 1 piece | 1 |
| Inari | 4 pieces | 3 |
| Futomaki | 6 pieces | 3 |
| Rainbow roll | 2 pieces | 1 |
| Tempura roll | 4 pieces | 3 |
| Tofu roll | 8 pieces | 3 |
| Tsunami roll | 5 pieces | 3 |
| Dragon roll | 4 pieces | 4 |
| Ocean crab roll | 8 pieces | 4 |
| Ebi-sushi (shrimp) | 4 oz | 1 |
| Kani-sushi (crab) | 3 oz | 1 |
| Sashimi (tuna fish) | 3 oz | 1 |
| Sashimi (beef) | 2 oz | 1 |
| Sashimi (squid) | 4 oz | 1 |
| Tekka-Maki (tuna fish) | 4 | 1 |
| Tamago-yaki (wrapped in | 2 | 1 |
| egg) | | |
| Chicken tempura | 3 oz | 3 |
| Shrimp teriyaki | 6 oz | 2 |
| Sushi and sashimi | 6 oz | 2 |
| Pork dumpling | 1 piece | 1 |
| Miso soup with tofu | 2 cups | 1 |
| Calamari salad | 5 oz | 2 |
| Edamame | 4 oz | 2 |
| Edamame salad | 6 oz | 1 |
| Harusame salad | 3 oz | 1 |
| Seabreeze salad | 3 oz | 1 |
| Chili sauce | 2 Tbsp | 1 |
| Pickled ginger | 4 oz | 1 |
| Wasabi (green horseradish) | 4 Tbsp | ½ |
| GREEK | | |
| Greek rice | 1 cup | 2 |
| Dolmades (stuffed grape | 2 pieces | 3 |
| leaves) | | |
| Greek salad without | 1 serving | 3 |
| dressing | | |
| Chicken Souvlaki (kebob) | 1 order | 3 |
| Tzatziki | 4 Tbsp | 1 |
| Hummus | 2 Tbsp | 1 |
| Marinated calamari (squid) | 1 starter portion | 2 |
| Keftethes (meatballs) | 2 | 1 |
| Taramasalata | 1 Tbsp | 1 |
| Melitzanosalata (aubergine | 3 Tbsp | 1 |
| puree salad) | | |
| Pita bread | ½ pita | 1 |
| Chicken soup and rice | 1 serving | 1 |
| Baked fish with tomatoes | 1 serving | 3 |
| and garlic | | |
| Lamb and pepper kebabs | 1 serving | 4 |
| Meatballs on skewers | ½ serving | 2 |
| Stifado (meat stew) | ½ serving | 3 |
| Meatballs | ½ serving | 3 |
| Moussaka | ½ serving | 4 |
| Spanakopita (spinach and | ½ serving | 4 |
| cheese pie) | | |
| Halva (sweetmeat made | 1 serving | 3 |
| with honey and sesame | | |
| seed) | | |
| Baklava (honey and walnut | 1 serving | 4 |
| pastry) | | |
| INDIAN | | |
| Cucumber raira | 4 Tbsp | 1 |
| Tomato sambal | 4 Tbsp | 1 |
| Mango chutney | 1 Tbsp | 1 |
| Poppadom | 1 | 1 |
| Lime pickle | 1 Tbsp | 1 |
| Onion bhaji | 1 | 2 |
| Vegetable samosa | Each | 3 |
| Keema madras | ½ serving | 3 |
| Meat samosa | Each | 4 |
| Tandoori chicken | 1 serving | 4 |
| Aloo gobi | 1 serving | 4 |
| Vegetable curry | 1 serving | 4 |
| Aloo saag | ½ serving | 3 |
| Vegetable biriyani | ½ serving | 3 |
| Lamb bhuna | ½ serving | 4 |
| Chicken tikka masala | ½ serving | 4 |
| Beef madras | ½ serving | 3 |
| Chicken curry | ½ serving | 4 |
| Rogan josh | ½ serving | 4 |
| Chicken dhansk | ½ serving | 4 |
| THAI | | |
| Fish cakes | 2 | 1 |
| Spring rolls | 1 | 1 |
| Yam talay (seafood salad | 1 serving | 2 |
| without dressing) | | |
| Satay and peanut sauce | 1 serving | 4 |
| Moo pad king (pork fried | 1 serving | 4 |
| with ginger) | | |
| Tom yam gung (hot & sour | 1 serving | 1 |
| soup with prawns) | | |
| Normai pad kai (pork with | 1 serving | 4 |
| bamboo shoots | | |
| Gai tome ke (chicken, | 1 serving | 1 |
| coconut and galangel soup) | 1 serving | 1 |
| Pla manow (fish with lemon | 1 serving | 4 |
| sauce) | | |
| Pad Thai (fried noodles) | ½ serving | 2 |
| Gai hoh bai teo (chicken | ½ serving | 2 |
| wrapped in bandan leaf) | | |
| Gang ped bhed yang (duck | ½ serving | 3 |
| curry) | | |
| Fresh pineapple | 1 serving | 1 |
| Som loy geow (oranges in | 1 serving | 2 |
| syrup) | | |
| Kow neuw mamuang | ½ serving | 4 |
| (mangoes with sticky rice) | | 4 |
| FRENCH | | |
| Mussels | 1 serving | 2 |
| Crudités with garlic | 1 serving | 3 |
| mayonnaise | | |
| Grilled goat's cheese salad | 1 serving | 3 |
| Pate de campagne | 1 serving | 3 |
| Snails | 1 serving | 3 |
| French onion soup | 1 serving | 4 |
| Shellfish bisque | ½ serving | 3 |
| Grilled dover sole | 1 serving | 3 |
| Grilled trout | 1 serving | 3 |
| Chicken chasseur | ½ serving | 3 |
| Steak au poivre | ½ serving | 3 |
| Steak with béarnaise sauce | ½ serving | 3 |
| Moules mariniere with | ½ serving | 3 |
| pommes frites | | |
| Coq au vin | ½ serving | 3 |
| Cassoulet | ½ serving | 3 |
| Beef bourguignon | ½ serving | 4 |
| Crème caramel | 1 serving | 2 |
| Chocolate mousse | 1 serving | 3 |
| Crème brulee | 1 serving | 4 |
| Crepe suzette | ½ serving | 2 |
| Chocolate gateau | ½ serving | 3 |
| Tarte au citron | ½ serving | 3 |
| Tarte tatin | ½ serving | 3 |
| Profiteroles | ½ serving | 4 |
| SPANISH | | |
| Bean soup | 8 oz | 2 |
| Setas alhomo (mushrooms | 1 serving | 2 |
| in garlic sauce) | | |
| Pimiento relleno (stuffed | 1 serving | 2 |
| pepper) | | |
| Patatas bravas (fried | 1 serving | 3 |
| potatoes in chili and garlic | | |
| sauce) | | |
| Calamari (deep fried squid) | 1 serving | 3 |
| Arroz con pollo (rice with | 1 serving | 2 |
| chicken) | | |
| Paella | 1 serving | 2 |
| Gambas al ajillo (garlic | 1 serving | 2 |
| shrimp) | | |
| Chorizo (spicy sausage) | ½ serving | 2 |
| Mussels | 1 serving | 3 |
| Tortilla (Spanish omelette) | 1 serving | 3 |
| Boquerones (whitebait) | 1 serving | 3 |
| Lamb stew | 1 serving | 4 |
| Albondigas (meatballs in | ½ serving | 2 |
| sauce) | | |
| Flan de almendras (almond | 1 serving | 3 |
| flan) | | |
| Flan de leche (baked | 1 serving | 4 |
| custard flan) | | |
| Arroz con leche (rice | ½ serving | 2 |
| pudding) | | |
| AMERICAN | | |
| Vegetable of the day | 1 cup | 1 |
| Baked potato with 1 Tbsp | 1 | 3 |
| sour cream | | |
| Mashed potatoes with gravy | ¾ cup | 2 |
| Chicken pot pie | ½ | 4 |
| Barbecue ribs | 1 serving | 4 |
| Chicken wings with | ½ serving | 3 |
| barbecue dip | | |
| Potato skins with sour | ½ serving | 4 |
| cream | | |
| Caesar salad | ½ serving | 3 |
| Chilli cheeseburger | ½ serving | 4 |
| Well done sirloin steak with | 3 oz | 3 |
| fries | | |
| Chocolate fudge cake | ½ serving | 2 |
| Banana split | ½ serving | 2 |
| Key lime pie with whipped | ½ serving | 3 |
| cream | | |
| Mississippi mud pie | ½ serving | 3 |
| Einstein Bros Bagels: | | |
| Bagel | 1 | 4 |
| Chicken noodle soup | 1 cup | 2 |
| Vegetable black bean soup | 1 cup | 2 |
| Cream of potato soup | 1 cup | 2 |
| Turkey chili with beans | 1 cup | 3 |
| soup | | |
| Honey butter or regular | 1 Tablespoon | 1 |
| butter | | |
| Hummus | 2 oz | 1 |
| Grape fruit spread | 1 oz | 1 |
| Cream cheese: whipped | 2 Tablespoons | 1 |
| plain reduced fat, or | | |
| whipped garden vegetable, | | |
| or blueberry or onion & | | |
| chive or plain | | |
| UK | | |
| Tomato soup | 1 serving | 2 |
| Prawn cocktail | 1 serving | 4 |
| Breaded mushrooms with | 1 serving | 4 |
| dip | | |
| Pate and toast | ½ serving | 2 |
| Venison in red wine | 1 serving | 3 |
| Pork and apple casserole | 1 serving | 4 |
| Lancashire hotpot | ½ serving | 2 |
| Shepherd's pie | ½ serving | 2 |
| Fish pie | ½ serving | 3 |
| Beef casserole | ½ serving | 3 |
| Well done rump steak with | 4 oz | 3 |
| chips | | |
| Roast beef with trimmings | ½ serving | 3 |
| Well done fillet steak and | 4 oz | 3 |
| chips | | |
| Beef Wellington | ½ serving | 3 |
| Sausage and mash | ½ serving | 4 |
| Toad in the hole | ½ serving | 4 |
| Gammon steak and chips | ½ serving | 4 |
| Beef stew with dumplings | ½ serving | 4 |
| Trifle | 1 serving | 3 |
| Lemon meringue pie | 1 serving | 4 |
| Apple pie and custard | ½ serving | 3 |
| Fruit crumble and custard | ½ serving | 3 |
| Treacle tart and custard | ½ serving | 3 |
| Cheesecake | ½ serving | 3 |
| Ice cream sundae | ½ serving | 3 |
| Bread and butter pudding | ½ serving | 3 |
| Sponge pudding and custard | ½ serving | 3 |
| STEAKHOUSE/RIBS | | |
| Roadhouse Grill's chicken | 8 oz | 3 |
| breast | | |
| Roadhouse Grill's chicken | 5 oz | 2 |
| breast | | |
| Roadhouse Grill's salad | 2 Tbsp | 2 |
| dressings Asian sesame | | |
| Roadhouse Grill's salad | 2 Tbsp | 1 |
| dressings Lite ranch | | |
| Filet mignon | 9 oz | 4 |
| Sirloin steak | 6 oz | 2 |
| Pork chops | 1 | 3 |
| Roadhouse Grill's Sirloin | 3 oz | 2 |
| steak | | |
| Roadhouse Grill's | 3 oz | 3 |
| tenderloin filet | | |
| Roadhouse Grill's chicken | 5 pieces | 4 |
| wings | | |
| Roadhouse Grill's | 4 oz | 3 |
| hamburger patty | | |
| Roadhouse Grill's prime rib | 4 oz | 4 |
| Roadhouse Grill's sirloin | 5 oz | 4 |
| Roadhouse Grill's | 4.5 oz | 4 |
| tenderloin filet | | |
| T-bone steak | 8 oz | 4 |
| THE FOOD COURT | | |
| Au Bon Pain Pesto chicken | 1 | 3 |
| salad | | |
| Au Bon Pain Thai chicken | ½ entree | 2 |
| Au Bon Pain everything | 1 | 4 |
| bagel | | |
| Au Bon Pain Thai chicken | ½ | 3 |
| salad with Thai peanut | | |
| dressing | | |
| Auntie Anne's original | 1 | 4 |
| pretzel | | |
| Great steak & potato fries, | ½ | 3 |
| small | | |
| Panda Express chicken with | ½ | 2 |
| mushrooms & rice | | |
| Sbarro cheese pizza | ¾ slice | 4 |
| Au Bon Pain chicken | ½ | 3 |
| Caesar wrap | | |
| Hamburger, loaded | ½ | 4 |
| Sbarro Spaghetti with sauce | ½ order | 3 |
| Auntie Anne's glazin' raisin | ½ | 3 |
| pretzel | | |
| Bagel with cream cheese | ½ | 3 |
| Great steak & potato fries | ½ regular | 3 |
| Chicken breast & wing, | ½ each | 3 |
| breaded & fried | | |
| Fish sandwich with tartar | ½ | 3 |
| sauce, not fried | | |
| Pot roast | 10 oz | 4 |
| Grilled or BBQ chicken | 1 entree | 3 |
| Chicken fingers | 2.5 | 4 |
| Chicken Caesar salad with | ½ | 4 |
| dressing | | |
| GENERAL FAST FOOD | | |
| Hamburger, plain | ½ large | 2 |
| Chicken nuggets | 6 | 3 |
| Thin French fries | 14 | 2 |
| Hamburger, regular, plain | 1 | 4 |
| Hot dog with bun, plain | 1 small | 3 |
| Movie theatre popcorn, no | Kid's size | 3 |
| butter | | |
| BEVERAGES | | |
| Starbucks: | | |
| Brewed coffee | 1 tall | free |
| Caffe Americano | 1 tall | free |
| Espresso | solo | free |
| Cappuccino with skim milk | 1 tall | 1 |
| Caffe latte with skim milk | 1 tall | 1 |
| Cafe mocha with skim milk, | 1 tall | 2 |
| no whip | | |
| Iced Cafe mocha with skim | 1 grande | 2 |
| milk, no whip | | |
| Tazo tea lemonade | 1 tall | 1 |
| Iced coffee shaken | 1 grande | 1 |
| Tazo iced tea, black or | 1 grande | 1 |
| passion | | |
| Frappuccino light coffee | 1 tall | 1 |
| Frappuccino light mocha | 1 grande | 2 |
| Frappuccino light caramel | 1 grande | 2 |
| Frappuccino coffee | 1 tall | 2 |
| Frappuccino espresso | 1 tall | 2 |
| Caramel macchiato with | 1 tall | 2 |
| skim milk | | |
| Espresso con Panna | Solo or doppio | 1 |
| Tazo chai tea latte with | 1 tall | 2 |
| skim milk | | |
| Iced Tazo chai tea latte with | 1 tall | 2 |
| skim milk | | |
| Vanilla crème with skim | 1 tall | 2 |
| milk, no whip | | |
| Vanilla latte with skim milk | 1 tall | 2 |
| Iced Vanilla latte with skim | 1 grande | 2 |
| milk | | |
| Iced caramel macchiato | 1 tall | 2 |
| with soy milk | | |
| Caramel Macchiato with | 16 oz | 2 |
| skim milk | | |
| Caffe mocha with skim | 16 oz | 4 |
| milk | | |
| White chocolate mocha | 16 oz | 4 |
| with skim milk | | |
| Coffee Frappuccino | 20 oz | 4 |
| McDonald's chocolate | 1 small | 4 |
| shake | | |
| Dunkin Donuts: | | |
| Coffee with cream & sugar | 1 | 1 |
| Coffee coolatta with skim | 1 | 3 |
| milk | | |
| Hot chocolate | 1 | 3 |
| Dunkaccino | 1 | 3 |
| Coffee coolatta with cream | 1 | 4 |
| SWEETS | | |
| Haagen-Dazs ® sorbet | 1 small scoop | 1 |
| Haagen-Dazs ® vanilla ice | 1 scoop | 3 |
| cream | | |
| TCBY ®, regular cone | 1 | 2 |
| Mrs. Fields ® milk | 1 | 3 |
| chocolate chip cookie | | |
| Mrs. Fields ® white chunk | 1 | 3 |
| macadamia cookie | | |
| Breyers ® fat free ice cream | 1 cup | 2 |
| Einstein's ® Raspberry low | 1 | 4 |
| fat scone | | |
| Dairy Queen ®: | | |
| Soft serve ice cream vanilla | ½ cup | 2 |
| or chocolate | | |
| Vanilla or chocolate | 1 small cone | 3 |
| Chocolate sundae | 1 small | 3 |
| Nonfat frozen yogurt | ½ cup | 1 |
| Yogurt strawberry sundae | 1 medium | 3 |
| Strawberry breeze | 1 small | 4 |
| Misty slush | 1 small | 3 |
| Fudge bar no sugar added | 1 | 1 |
| Vanilla orange bar, no sugar | 1 | 1 |
| added | | |
| Ice cream sandwich | 1 | 2 |
| Chocolate dilly bar | 1 | 2 |
| Au Bon Pain ® low fat triple | 1 | 3 |
| berry muffin | | |
| Entenmann's ® fat free | 1/10 | 1 |
| chocolate loaf cake | | |
| Dunkin Donuts ®: | | |
| Chocolate glazed cake | 1 donut | 4 |
| Apple ‘n spice | 1 | 2 |
| Chocolate frosted | 1 | 2 |
| Bavarian Kreme | 1 | 2 |
| Lemon | 1 | 2 |
| Jelly filled | 1 | 2 |
| Chocolate glazed | 3 | 2 |
| munchkins | | |
| Glazed munchkins | 3 | 2 |
| Plain munchkins | 4 | 2 |
| Boston Kreme | 1 | 3 |
| Old fashioned cake | 1 | 3 |
| Sugared cake | 1 | 3 |
| Glazed fritter | 1 | 3 |
| Coffee roll | 1 | 3 |
| Powdered cake | 1 | 3 |
| Glazed cake | 1 | 3 |
| Vanilla Kreme filled | 1 | 3 |
| Cherry Danish muffin | 1 | 3 |
| Cinnamon Danish muffin | 1 | 4 |

1. A method of weight management comprising one or both of weight loss and target weight maintenance, said method comprising:
consuming a plurality of meals per day, wherein the total daily caloric content of said meals comprises about 41 to 49% carbohydrates selected from fiber rich foods containing at least about 2 grams of dietary fiber for each 90 calorie serving of said fiber rich food.

2. The method recited in claim 1 wherein the total daily caloric content of said meals further comprises about 22 to 28% protein selected from lean protein sources having less than about 5 grams of fat per ounce of the lean protein source.

3. The method recited in claim 1 wherein the total daily caloric content of said meals further comprises about 26 to 34% fats selected from monounsaturated and polyunsaturated fats.

4. The method recited in claim 1 wherein the daily fiber intake is at least about 25 grams per day for women and at least about 35 grams per day for men.

5. The method recited in claim 1 wherein the total daily caloric content of said meals comprises about 45% carbohydrates, about 25% lean protein and about 30% fats.

6. The method recited in claim 5 wherein said lean proteins comprises primary proteins and secondary proteins and the total daily caloric content of said meals comprises consuming portions of food from each of primary proteins, secondary proteins, milk or yogurt products, starch, fruit, vegetables and fats.

7. The method recited in claim 6 wherein four meals per day are consumed.

8. The method recited in claim 7 further wherein each serving of food comprises about 90 to 100 calories.

9. The method recited in claim 8 wherein the total daily caloric intake for men initially comprises five servings of starch, two servings of fruit, two servings of milk or yogurt products, four servings of fats, two servings of primary proteins, two servings of secondary proteins and two servings of vegetables.

10. The method recited in claim 9 wherein the total daily caloric intake for men further comprises up to four dividend servings of food or beverages having in total a daily caloric content of no more than about 80 calories.

11. The method recited in claim 10 further comprising establishing a desired weekly weight loss target and adjusting the total caloric intake until said weekly weight loss target is achieved.

12. The method recited in claim 11 wherein said step of adjusting the total caloric intake comprises one of:
adding one daily serving of any one of said milk or yogurt products, starch, fruit or vegetables per week until said weight loss target is achieved when weekly weight loss exceeds said target; and,
subtracting one daily serving of any one of said primary proteins, secondary proteins or fats per week until said weight loss target is achieved when weekly weight loss is less than said target.

13. The method recited in claim 8 wherein the total daily caloric intake for women initially comprises four servings of starch, two servings of fruit, two servings of milk or yogurt products, three servings of fats, two servings of primary proteins, one serving of secondary proteins and one serving of vegetables.

14. The method recited in claim 13 wherein the total daily caloric intake for women further comprises up to four dividend servings of food or beverages having in total a daily caloric content of no more than about 80 calories.

15. The method recited in claim 14 further comprising establishing a desired weekly weight loss target and adjusting the total caloric intake until said weekly weight loss target is achieved.

16. The method recited in claim 15 wherein said step of adjusting the total caloric intake comprises one of:
adding one daily serving of any one of said milk or yogurt products, starch, fruit or vegetables per week until said weight loss target is achieved when weekly weight loss exceeds said target; and,
subtracting one daily serving of any one of said primary proteins, secondary proteins or fats per week until said weight loss target is achieved when weekly weight loss is less than said target.

17. The method recited in claim 1 further comprising engaging in physical activity sufficient to use at least about 1500 calories per week.

18. The method recited in claim 1 further comprising engaging in physical activity sufficient to use at least about 2000 calories per week.

19. The method recited in claim 1 wherein the total daily caloric intake for men ranges from about 1600 to about 2000 calories.

20. The method recited in claim 1 wherein the total daily caloric intake for men is initially set at about 1800 calories for the first week of the diet and thereafter optionally adjusted in the event that a weekly weight loss target is not achieved.

21. The method recited in claim 20 wherein the total daily caloric intake is adjusted in increments of one serving of a food group per week until the weekly weight loss target is achieved.

22. The method recited in claim 1 wherein the total daily caloric intake for women ranges from about 1200 to about 1500 calories.

23. The method recited in claim 1 wherein the total daily caloric intake for women is initially set at about 1400 calories for the first week of the diet and thereafter optionally adjusted in the event that a weekly weight loss target is not achieved.

24. The method recited in claim 23 wherein the total daily caloric intake is adjusted in increments of one serving of a food group per week until the weekly weight loss target is achieved.

25. The method recited in claim 1 further comprising drinking about 6-8 eight ounce servings of water per day.

26. The method recited in claim 25 further comprising drinking an additional eight ounce serving of water per day for every twenty-five pounds over a recommended weight target.

27. A method of counseling an overweight individual regarding weight loss and weight management comprising:
counseling the individual to consume a first group of foods comprising low fat, fiber rich carbohydrates comprising about 41 to 49% of the total daily calories consumed, excluding any calories from fat if present in the first group of food;
counseling the individual to select carbohydrates having at least about 2 grams of fiber for every 90 calories of the carbohydrate
counseling the individual to consume a second group of foods comprising lean protein in an amount comprising about 22 to 28% of total calories consumed, excluding any calories from fat if present in the second group of foods; and
counseling the individual to consume heart-healthy fats in an amount comprising about 26 to 34% of total calories consumed.

28. The method recited in claim 27 further comprising counseling the individual to engage in activity sufficient to bum at least about 1500 calories on average per week.

29. The method recited in claim 27 further comprising counseling the individual to liberally partake of a group of foods comprising foods having no or minimal caloric value.

30. The method recited in claim 27 further comprising providing the individual with a plurality of menu formats until a desired weight objective is achieved, and thereafter to maintain a desired weight range.

31. The method recited in claim 27 further comprising teaching component food types to be structured into up to four meals each day, teaching serving sizes effective for achieving a desired weight objective, teaching which proteins are acceptable proteins effective for achieving a desired weight objective, teaching which fats on are acceptable fats effective for achieving a desired weight objective, and teaching which carbohydrates are acceptable carbohydrates effective for achieving a desired weight objective.

32. The method recited in claim 31 wherein teaching which proteins are acceptable proteins comprises teaching the use of lean proteins comprised of primary proteins and secondary proteins.

33. The method recited in claim 31 wherein teaching which fats are acceptable comprises teaching the use of monounsaturated and polyunsaturated fats.

34. The method recited in claim 31 further comprising establishing a desired weekly weight loss target and counseling the individual to adjust the total daily calories consumed until said weekly weight loss target is achieved.

35. The method recited in claim 34 wherein the weekly weight loss target is two pounds per week until the total weight loss objective is achieved.

36. The method recited in claim 27 further comprising counseling the individual that the daily caloric intake of foods includes at least one serving of each of a primary protein, secondary protein, milk or yogurt product, starch, fruit, vegetable and fat.

37. The method recited in claim 36 further comprising counseling the individual to consume four meals per day.

38. The method recited in claim 37 further comprising counseling the individual that each serving of food comprises about 90 calories.

39. The method recited in claim 38 further comprising counseling a male individual to select as his total daily caloric intake five servings of starch, two servings of fruit, two servings of milk or yogurt products, four servings of fats, two servings of primary proteins, two servings of secondary proteins and two servings of vegetables.

40. The method recited in claim 39 further comprising counseling a male individual to additionally select up to four dividend foods selected from foods and beverages substantially devoid of caloric value, foods and beverages in quantities sufficiently small that such foods and beverages are substantially devoid of caloric value, fat free condiments and combinations thereof.

41. The method recited in claim 40 further comprising establishing a desired weekly weight loss target for a male individual and counseling the male individual to consume a total daily caloric intake of about 1800 calories for the first week of the diet and thereafter optionally to adjust the total daily caloric intake in the event that the desired weekly weight loss target is not achieved.

42. The method recited in claim 41 wherein the total daily caloric intake is adjusted in increments of one serving of a food group per week until the weekly weight loss target is achieved.

43. The method recited in claim 38 further comprising counseling a female individual to select as her total daily caloric intake four servings of starch, two servings of fruit, two servings of milk or yogurt products, three servings of fats, two servings of primary proteins, one serving of secondary proteins and one serving of vegetables.

44. The method recited in claim 43 further comprising counseling a female individual to additionally select up to four dividend foods selected from selected from foods and beverages substantially devoid of caloric value, foods and beverages in quantities sufficiently small that such foods and beverages are substantially devoid of caloric value, fat free condiments and combinations thereof.

45. The method recited in claim 44 further comprising establishing a desired weekly weight loss target for a female individual and counseling the female individual to consume a total daily caloric intake of about 1400 calories for the first week of the diet and thereafter optionally to adjust the total daily caloric intake in the event that the desired weekly weight loss target is not achieved.

46. The method recited in claim 45 wherein the total daily caloric intake is adjusted in increments of one serving of a food group per week until the weekly weight loss target is achieved.

47. The method recited in claim 27 further comprising counseling the individual to drink about 6-8 eight ounce servings of water per day.

48. The method recited in claim 47 further comprising drinking an additional eight ounce serving of water per day for every twenty-five pounds over a recommended weight target.

49. A method of counseling an overweight individual regarding weight loss and weight management comprising:
establishing a desired weekly weight loss target for an overweight individual;
counseling the individual to consume a first group of foods comprising low fat, fiber rich carbohydrates comprising about 45% of the total daily calories consumed, excluding any calories from fat if present in the first group of food;
counseling the individual to select carbohydrates having at least about 2 grams of fiber for every 90 calories of the carbohydrate
counseling the individual to consume a second group of foods comprising lean protein in an amount comprising about 25% of total calories consumed, excluding any calories from fat if present in the second group of foods;
counseling the individual to consume heart-healthy fats in an amount comprising about 30% of total calories consumed;
counseling the individual that the daily caloric intake of foods includes at least one serving of each of a primary protein, secondary protein, milk or yogurt product, starch, fruit, vegetable and fat;
adjusting the total caloric intake for the individual in the event that said weekly weight loss target is not being achieved until said weekly weight loss target is achieved by
adding one daily serving of any one of said milk or yogurt products, starch, fruit or vegetables per week until said weight loss target is achieved when weekly weight loss exceeds said target; and,
subtracting one daily serving of any one of said primary proteins, secondary proteins or fats per week until said weight loss target is achieved when weekly weight loss is less than said target;
counseling the individual to drink at least about 6-8 eight ounce servings of water per day and to drink an additional eight ounces for each 25 pounds by which the individual weighs over a target weight for that individual;
counseling the individual to liberally partake of a group of foods comprising foods having no or minimal caloric value;
counseling the individual to engage in activity sufficient to bum at least about 1500 calories on average per week; and
providing the individual with a plurality of daily menu formats until a desired weight objective is achieved.
